(19) United States
(12) Patent Application Publication
(10) Pub. No.: US 2020/0405529 A1
(43) Pub. Date: Dec. 31, 2020

(54) THERMAL SYSTEM WITH USER INTERFACE CUSTOMIZATION

(71) Applicant: Stryker Corporation, Kalamzoo, MI (US)

(72) Inventors: Gregory S. Taylor, Kalamazoo, MI (US); Marco Constant, Johnson City, TN (US); Christopher John Hopper, Kalamazoo, MI (US); Gideon P. Brewer, Grand Rapids, MI (US)

(21) Appl. No.: 16/912,244

(22) Filed: Jun. 25, 2020

Related U.S. Application Data
(60) Provisional application No. 62/868,183, filed on Jun. 28, 2019.

(51) Int. Cl. A61F 7/00; G08B 3/10

(52) U.S. Cl. CPC A61F 7/0085; G08B 3/10; A61F 2007/0093; A61F 2007/0086; A61F 2007/0096; A61F 2007/0056

(57) ABSTRACT

A thermal control unit for controlling a patient's temperature includes a fluid outlet for delivering temperature-controlled fluid to a patient, a fluid inlet for receiving the fluid back, a pump, a heat exchanger, a controller, a patient temperature port for receiving patient temperature readings, a memory, a user interface, and an auxiliary input. In some embodiments, the controller is adapted to display an indication on the display identifying a type of auxiliary sensor that the user should couple to the auxiliary input in order to carry out the thermal therapy session. The memory may contain a set of alarm conditions and the controller may be adapted to allow a user to customize the set of alarm conditions. The controller may also display a combined graph showing both patient temperature readings and auxiliary input readings with respect to time.

### CROSS-REFERENCE TO RELATED APPLICATIONS

This application claims priority to U.S. provisional patent application Ser. No. 62/868,183 filed Jun. 28, 2019, by inventors Gregory S. Taylor et al. and entitled THERMAL SYSTEM WITH USER INTERFACE CUSTOMIZATION, the complete disclosure of which is incorporated herein by reference.

### BACKGROUND

The present disclosure relates to a thermal control system for controlling the temperature of circulating fluid that is delivered to one or more thermal devices positioned in contact with a patient.

Thermal control systems are known in the art for controlling the temperature of a patient by providing a thermal control unit that supplies temperature-controlled fluid to one or more thermal pads or catheters positioned in contact with a patient. The thermal control unit includes one or more heat exchangers for controlling the temperature of the fluid and a pump that pumps the temperature-controlled fluid to the pad(s) and/or catheter. After passing through the pad(s) and/or catheter, the fluid is returned to the thermal control unit where any necessary adjustments to the temperature of the returning fluid are made before being pumped back to the pad(s) and/or catheter. In some instances, the temperature of the fluid is controlled to a static target temperature, while in other instances the temperature of the fluid is varied as necessary in order to automatically effectuate a target patient temperature.

Thermal control units typically include a user interface adapted to allow the user to input information for using the thermal control unit, as well as for displaying information useful to the user of the thermal control unit. Thermal treatment sessions in which the thermal control unit is utilized for controlling the patient's temperature can be used for a variety of different reasons, such as, but not limited to, cooling patients after cardiac arrest, cooling patents for neurosurgery (or other types of surgery), cooling patients who have experience a neurotrauma, treating fevers, and still other uses. In addition to the various uses of thermal control units, different individuals may interact with the thermal control unit (e.g. doctors, nurses, technicians). It is therefore desirable to have a thermal control unit that is easily usable for different treatments and/or for different users.

### SUMMARY

The present disclosure is directed to an improved thermal control unit that is adapted to be more easily and efficiently utilized by different individuals and/or for different types of treatment. The thermal control unit may be configured to include an auxiliary sensor input and a display that automatically informs the user what type of auxiliary sensor should be used with a particular thermal therapy session. The thermal control unit may also, or alternatively, include a plurality of default alarm conditions that are customizable by the users. The customization of the default alarm conditions may also, or alternatively, include customizing one or more aspects of the alarm, such as, but not limited to, the volume, tone, duration, etc. of the alarm. In some embodiments, the thermal control unit is adapted to plot an output of the auxiliary sensor on the display of the thermal control unit on a graph that also includes other patient data, such as, but not limited to, a plot of the patient's temperature with respect to time. Still further, the thermal control unit may be configured to allow a user to customize the treatment profile. In all cases, the thermal control unit may store multiple sets of customization data and automatically implement one or more of the sets of customization data based upon a particular user of the thermal control unit, a particular type of therapy to be applied by the thermal control unit, a particular location of the thermal control unit within a healthcare facility, and/or other factors.

According to one embodiment of the present disclosure, a thermal control unit is provided for controlling a patient's temperature during a thermal therapy session. The thermal control unit includes a fluid outlet, a fluid inlet, a circulation channel, a pump, a heat exchanger, a fluid temperature sensor, a patient temperature sensor, an auxiliary input, a display, and a controller. The fluid outlet is adapted to fluidly couple to a fluid supply line and the fluid inlet is adapted to fluidly couple to a fluid return line. The circulation channel is coupled to the fluid inlet and the fluid outlet and the pump circulates the fluid through the circulation channel from the fluid inlet to the fluid outlet. The heat exchanger is adapted to add or remove heat from the fluid circulating in the circulation channel. The fluid temperature sensor is adapted to sense a temperature of the fluid and the patient temperature sensor port is adapted to receive patient temperature readings from a patient temperature sensor. The auxiliary input is adapted to receive an output from an auxiliary sensor. The controller is adapted to control the heat exchanger in order to control the patient's temperature and to display an indication on the display identifying a type of auxiliary sensor the user should couple to the auxiliary input in order to carry out the thermal therapy session.

According to other aspects of the present disclosure, the auxiliary sensor may be adapted to detect at least one of the following characteristics of the patient: an end tidal carbon dioxide (ETCO₂) level, an oxygen saturation (SpO₂) level, a respiration rate, a blood pressure, a heart rate, an electrolyte level, a pulse wave velocity, a bioimpedance, an electrocardiogram, or a rate of temperature change.

In some embodiments, the thermal control unit also includes a therapy-type input adapted to receive an input from the user indicating a therapy type for the thermal therapy session. In such embodiments, the controller is adapted to automatically select the type of auxiliary sensor based on the therapy-type input. The therapy-type input may be a control on a user interface of the thermal control unit, and the therapy type may include any one or more of the following types of therapy: a cardiac arrest therapy, a neuro-trauma therapy, a neurosurgery therapy, a fever therapy, or a pediatric therapy.

In some embodiments, the thermal control unit also includes a location input adapted to receive an input indicating a location of the thermal control unit within a healthcare facility. In such embodiments, the controller may be adapted to automatically select the type of auxiliary sensor based on the location input. The location input may be a screen on the display in which the user enters the location of the thermal control unit.

In some embodiments, the controller is further adapted to display a graph that includes both a plot of patient temperature readings from the patient temperature sensor port plotted with respect to time and a plot of auxiliary readings from the auxiliary input plotted with respect to time.

In some embodiments, the thermal control unit further includes a user input adapted to receive user data identifying a user of the thermal control unit. In such embodiments, the controller is adapted to automatically select the type of auxiliary sensor based on the user data.

The controller of the thermal control unit, in some embodiments, is adapted to use the output from the auxiliary sensor to control the temperature of the circulating fluid.

The thermal control unit, in some embodiments, further includes a memory containing a default set of alarm conditions. In such embodiments, the controller is further configured to allow a user to customize the default set of alarm conditions. The set of default alarm conditions may include any one or more of the following: a patient temperature sensor disconnection, a high fluid temperature, a low fluid temperature, a low fluid flow rate, a pause in therapy, a low fluid level, a patient temperature deviation, or a patient temperature sensor malfunction.

In some embodiments, the memory may also, or alternatively, include a plurality of alarm characteristics for each of the alarm conditions. In such embodiments, the controller is further adapted to allow the user to customize the plurality of alarm characteristics for each of the alarm conditions. The alarm characteristics may include any one or more of the following: an on/off setting, a tone setting, a priority setting, a repeat/non-repeat setting, a delay between repeats setting, an audio pause setting, and a pause duration setting.

In some embodiments, the thermal control unit includes a memory containing a therapy profile. In such embodiments, the controller is configured to follow the therapy profile during the thermal therapy session. Further, in such embodiments, the controller may be adapted to allow a user to customize the therapy profile and to store the customized therapy profile in the memory. The therapy profile may define any of the following parameters: a target temperature for cooling the patient, a cooling rate for the patient, an amount of time the patient is maintained at a temperature, a warming rate for the patient, or a target temperature for warming the patient.

In some embodiments, the auxiliary sensor is a potassium sensor adapted to detect a level of potassium in the patient, and the controller is adapted to display the potassium level on the display.

According to another embodiment of the present disclosure, a thermal control unit is provided for controlling a patient's temperature during a thermal therapy session. The thermal control unit includes a fluid outlet, a fluid inlet, a circulation channel, a pump, a heat exchanger, a fluid temperature sensor, a patient temperature sensor, a memory, a display, and a controller. The fluid outlet is adapted to fluidly couple to a fluid supply line and the fluid inlet is adapted to fluidly couple to a fluid return line. The circulation channel is coupled to the fluid inlet and the fluid outlet and the pump circulates the fluid through the circulation channel from the fluid inlet to the fluid outlet. The heat exchanger is adapted to add or remove heat from the fluid circulating in the circulation channel. The fluid temperature sensor is adapted to sense a temperature of the fluid and the patient temperature sensor port is adapted to receive patient temperature readings from a patient temperature sensor. The memory contains a set of alarm conditions and the controller is adapted to control the heat exchanger in order to control the patient's temperature. The controller is further adapted to issue an alarm in response to detecting any one of the alarm conditions in the set of alarm conditions, as well as to allow a user to customize the set of alarm conditions.

In some embodiments, the controller is adapted to allow the user to customize the set of alarm conditions by adding an alarm condition to, and/or subtracting an alarm condition from, the set of alarm conditions.

The alarm conditions may include any one or more of the following: a patient temperature sensor disconnection, a high fluid temperature, a low fluid temperature, a low fluid flow rate, a pause in therapy, a low fluid level, a patient temperature deviation, or a patient temperature sensor malfunction.

In some embodiments, the memory further includes a plurality of alarm characteristics for each of the plurality of alarm conditions, and the controller is further adapted to allow the user to customize the plurality of alarm characteristics for each of the plurality of alarms conditions. The alarm characteristics may include an on/off setting, a tone setting, a priority setting, a repeat/non-repeat setting, a delay between repeats setting, an audio pause setting, and/or a pause duration setting.

In some embodiments, the memory also include a therapy profile, and the controller is configured to follow the therapy profile during the thermal therapy session. The controller may be adapted to allow the therapy profile to be customized by the user, and to store the customized therapy profile in memory.

In some embodiments, the thermal control unit further includes an auxiliary input adapted to receive an output from an auxiliary sensor, and the controller is further configured to display an indication on the display identifying a type of auxiliary sensor the user should couple to the auxiliary input in order to carry out the thermal therapy session.

The auxiliary sensor may be adapted to detect any of the following characteristics of the patient: an end tidal carbon dioxide (ETCO₂) level, an oxygen saturation (SpO₂) level, a respiration rate, a blood pressure, a heart rate, an electrolyte level, a pulse wave velocity, a bioimpedance, an electrocardiogram, or a rate of temperature change.

In some embodiments, the thermal control unit includes a therapy-type input adapted to receive an input from the user indicating a therapy type for the thermal therapy session, and the controller is adapted to automatically select the type of auxiliary sensor based on the therapy-type input.

The thermal control unit may also, or alternatively, include a user input adapted to receive user data identifying a user of the thermal control unit. In such embodiments, the controller is adapted to automatically select the type of auxiliary sensor based on the user data.

According to another embodiment of the present disclosure, a thermal control unit is provided for controlling a patient's temperature during a thermal therapy session. The thermal control unit includes a fluid outlet, a fluid inlet, a circulation channel, a pump, a heat exchanger, a fluid temperature sensor, a patient temperature sensor, an auxiliary input, a display, and a controller. The fluid outlet is adapted to fluidly couple to a fluid supply line and the fluid inlet is adapted to fluidly couple to a fluid return line. The circulation channel is coupled to the fluid inlet and the fluid outlet and the pump circulates the fluid through the circulation channel from the fluid inlet to the fluid outlet. The heat exchanger is adapted to add or remove heat from the fluid circulating in the circulation channel. The fluid temperature sensor is adapted to sense a temperature of the fluid and the patient temperature sensor port is adapted to receive patient temperature readings from a patient temperature sensor. The auxiliary input is adapted to receive an output from an auxiliary sensor. The controller is adapted to control the heat exchanger in order to control the patient's temperature. The controller is further adapted to display a graph that includes both a plot of patient temperature readings from the patient temperature sensor port plotted with respect to time and a plot of auxiliary readings from the auxiliary input plotted with respect to time.

In some embodiments, the auxiliary sensor is an electrolyte sensor adapted to detect a level of an electrolyte in the patient, or an end tidal carbon dioxide (ETCO₂) sensor adapted to detect an ETCO₂ level of the patient.

In some embodiments, the auxiliary sensor is an electrocardiograph (ECG) sensor adapted to detect ECG signals from the patient, and the controller is further adapted to determine a potassium level of the patient from the ECG signals.

In some embodiments, the thermal control unit further includes a second auxiliary input adapted to receive an output from a second auxiliary sensor, and the controller is further adapted to include a plot of readings from the second auxiliary sensor on the graph plotted with respect to time. The auxiliary sensor may be adapted to detect any of the following characteristics of the patient: an end tidal carbon dioxide (ETCO₂) level, an oxygen saturation (SpO₂) level, a respiration rate, a blood pressure, a heart rate, an electrolyte level, a pulse wave velocity, a bioimpedance, an electrocardiogram, or a rate of temperature change.

In some embodiments, the controller is further adapted to allow a user to customize a therapy profile and to store the customized therapy profile in the memory. The therapy profile may define any one or more of the following: a target temperature for cooling the patient, a cooling rate for the patient, an amount of time the patient is maintained at a temperature, a warming rate for the patient, or a target temperature for warming the patient.

Before the various embodiments disclosed herein are explained in detail, it is to be understood that the claims are not to be limited to the details of operation or to the details of construction, nor to the arrangement of the components set forth in the following description or illustrated in the drawings. The embodiments described herein are capable of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the claims to any specific order or number of components. Nor should the use of enumeration be construed as excluding from the scope of the claims any additional steps or components that might be combined with or into the enumerated steps or components.

### BRIEF DESCRIPTION OF THE DRAWINGS

FIG. 1 is a perspective view of a thermal control system according to one aspect of the present disclosure shown applied to a patient on a patient support apparatus;

FIG. 2 is a perspective view of a thermal control unit of the thermal control system of FIG. 1;

FIG. 3 is a block diagram of a first embodiment of the thermal control system of FIG. 1;

FIG. 4 is an example of an alarm condition selection screen displayable on a display of the thermal control unit;

FIG. 5 is an example of an alarm customization screen displayable on the thermal control unit;

FIG. 6 is an example of a location selection screen displayable on the thermal control unit;

FIG. 7 is an example of a user selection screen displayable on the thermal control unit;

FIG. 8 is an example of a therapy selection screen displayable on the thermal control unit;

FIG. 9 is an example of an auxiliary sensor instruction screen displayable on the thermal control unit;

FIG. 10 is an example of a first therapy profile editing screen displayable on the thermal control unit;

FIG. 11 is an example of a second therapy profile editing screen displayable on the thermal control unit;

FIG. 12 is a diagram of a first example of customization records that may be maintained in a memory of the thermal control unit;

FIG. 13 is a diagram of a second example of customization records that may be maintained in the memory of the thermal control unit; and

FIG. 14 is an example of a graph displayable on the thermal control unit that shows both current patient temperature readings plotted with respect to time and potassium levels of the patient plotted with respect to time.

### DETAILED DESCRIPTION OF THE EMBODIMENTS

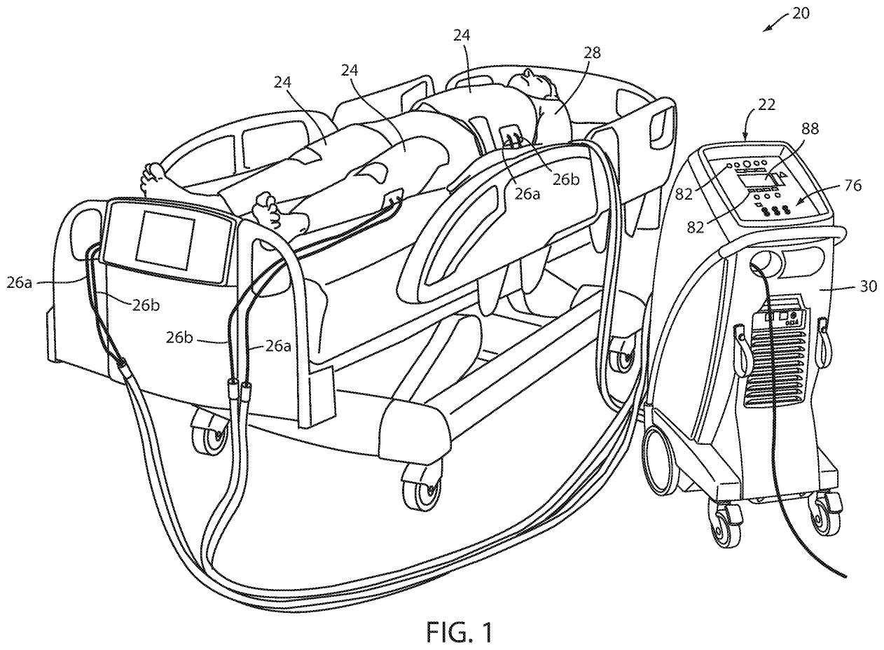

A thermal control system 20 according to one embodiment of the present disclosure is shown in FIG. 1. Thermal control system 20 is adapted to control the temperature of a patient 28, which may involve raising, lowering, and/or maintaining the patient's temperature. Thermal control system 20 includes a thermal control unit 22 coupled to one or more thermal therapy devices 24. The thermal therapy devices 24 are illustrated in FIG. 1 to be thermal pads, but it will be understood that thermal therapy devices 24 may take on other forms, such as, but not limited to, blankets, vests, patches, caps, catheters, or other structures that receive temperature-controlled fluid. For purposes of the following written description, thermal therapy devices 24 will be referred to as thermal pads 24, but it will be understood by those skilled in the art that this terminology is used merely for convenience and that the phrase “thermal pad” is intended to cover all of the different variations of thermal therapy devices 24 mentioned above (e.g. blankets, vests, patches, caps, catheters, etc.) and variations thereof.

Thermal control unit 22 is coupled to thermal pads 24 via a plurality of hoses 26. Thermal control unit 22 delivers temperature-controlled fluid (such as, but not limited to, water or a water mixture) to the thermal pads 24 via the fluid supply hoses 26a. After the temperature-controlled fluid has passed through thermal pads 24, thermal control unit 22 receives the temperature-controlled fluid back from thermal pads 24 via the return hoses 26b.

In the embodiment of thermal control system 20 shown in FIG. 1, three thermal pads 24 are used in the treatment of patient 28. A first thermal pad 24 is wrapped around a patient's torso, while second and third thermal pads 24 are wrapped, respectively, around the patient's right and left legs. Other configurations can be used and different numbers of thermal pads 24 may be used with thermal control unit 22, depending upon the number of inlet and outlet ports that are included with thermal control unit 22. By controlling the temperature of the fluid delivered to thermal pads 24 via supply hoses 26a, the temperature of the patient 28 can be controlled via the close contact of the pads 24 with the patient 28 and the resultant heat transfer therebetween.

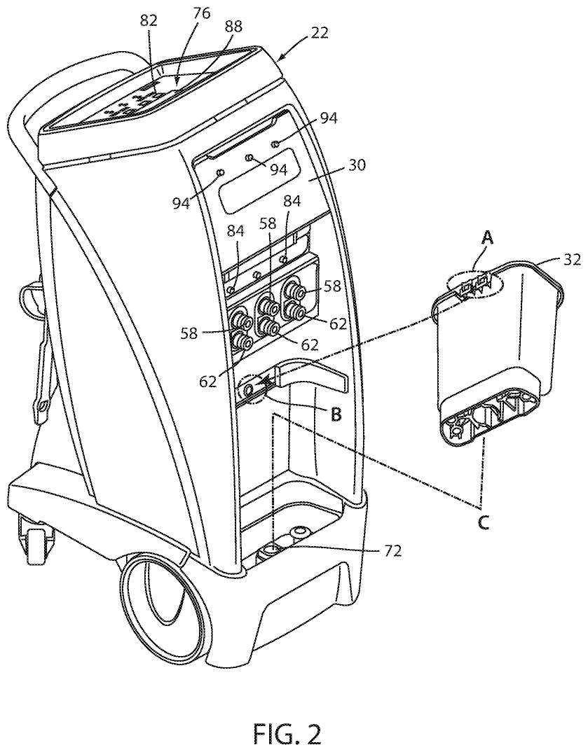

As shown more clearly in FIG. 2, thermal control unit 22 includes a main body 30 to which a removable reservoir 32 may be coupled and uncoupled. Removable reservoir 32 is configured to hold the fluid that is to be circulated through thermal control unit 22 and the one or more thermal pads 24. By being removable from thermal control unit 22, reservoir 32 can be easily carried to a sink or faucet for filling and/or dumping of the water or other fluid. This allows users of thermal control system 20 to more easily fill thermal control unit 22 prior to its use, as well as to drain thermal control unit 22 after use.

As can also be seen in FIG. 2, thermal control unit 22 includes a plurality of outlet ports 58 (three in the particular example of FIG. 2), a plurality of inlet ports 62 (three in this particular example). Outlet ports 58 are adapted to fluidly couple to supply hoses 26 and inlet ports are adapted to fluidly couple to return hoses 26b. Thermal control unit 22 also includes a plurality of patient temperature probe ports 84, a plurality of auxiliary ports 94, and a user interface 76 having a plurality of dedicated controls 82 and a display 88. The patient temperature probe ports 84, auxiliary ports 94, and user interface 76 are described in more detail below.

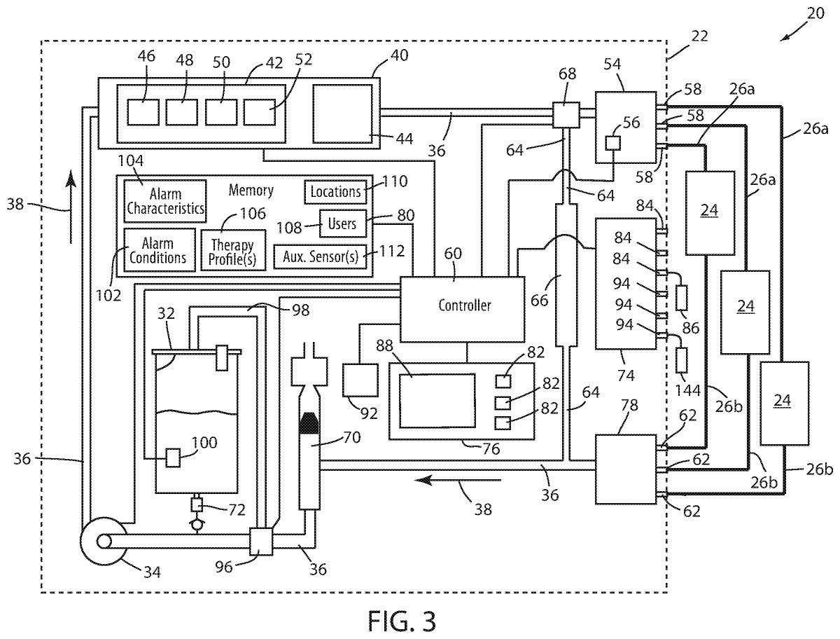

As shown in FIG. 3, thermal control unit 22 includes a pump 34 for circulating fluid through a circulation channel 36. Pump 34, when activated, circulates the fluid through circulation channel 36 in the direction of arrows 38 (clockwise in FIG. 3). Starting at pump 34 the circulating fluid first passes through a heat exchanger 40 that adjusts, as necessary, the temperature of the circulating fluid. Heat exchanger 40 may take on a variety of different forms. In some embodiments, heat exchanger 40 is a thermoelectric heater and cooler. In the embodiment shown in FIG. 3, heat exchanger 40 includes a chiller 42 and a heater 44. Further, in the embodiment shown in FIG. 3, chiller 42 is a conventional vapor-compression refrigeration unit having a compressor 46, a condenser 48, an evaporator 50, an expansion valve (not shown), and a fan 52 for removing heat from the compressor 46. Heater 44 is a conventional electrical resistance-based heater. Other types of chillers and/or heaters may be used.

After passing through heat exchanger 40, the circulating fluid is delivered to an outlet manifold 54 having an outlet temperature sensor 56 and a plurality of outlet ports 58. Temperature sensor 56 is adapted to detect a temperature of the fluid inside of outlet manifold 54 and report it to a controller 60. Outlet ports 58 are coupled to supply hoses 26a. Supply hoses 26a are coupled, in turn, to thermal pads 24 and deliver temperature-controlled fluid to the thermal pads 24. The temperature-controlled fluid, after passing through the thermal pads 24, is returned to thermal control unit 22 via return hoses 26b. Return hoses 26b couple to a plurality of inlet ports 62. Inlet ports 62 are fluidly coupled to an inlet manifold 78 inside of thermal control unit 22.

Thermal control unit 22 also includes a bypass line 64 fluidly coupled to outlet manifold 54 and inlet manifold 78 (FIG. 3). Bypass line 64 allows fluid to circulate through circulation channel 36 even in the absence of any thermal pads 24 or hoses 26a being coupled to any of outlet ports 58. In the illustrated embodiment, bypass line 64 includes an optional filter 66 that is adapted to filter the circulating fluid. If included, filter 66 may be a particle filter adapted to filter out particles within the circulating fluid that exceed a size threshold, or filter 66 may be a biological filter adapted to purify or sanitize the circulating fluid, or it may be a combination of both. In some embodiments, filter 66 is constructed and/or positioned within thermal control unit 22 in any of the manners disclosed in commonly assigned U.S. patent application Ser. No. 62/404,676 filed Oct. 11, 2016, by inventors Marko Kostic et al. and entitled THERMAL CONTROL SYSTEM, the complete disclosure of which is incorporated herein by reference.

The flow of fluid through bypass line 64 is controllable by way of a bypass valve 68 positioned at the intersection of bypass line 64 and outlet manifold 54 (FIG. 3). When open, bypass valve 68 allows fluid to flow through circulation channel 36 to outlet manifold 54, and from outlet manifold 54 to the connected thermal pads 24. When closed, bypass valve 68 stops fluid from flowing to outlet manifold 54 (and thermal pads 24) and instead diverts the fluid flow along bypass line 64. In some embodiments, bypass valve 68 may be controllable such that selective portions of the fluid are directed to outlet manifold 54 and along bypass line 64. In some embodiments, bypass valve 68 is controlled in any of the manners discussed in commonly assigned U.S. patent application Ser. No. 62/610,319, filed Dec. 26, 2017, by inventors Gregory Taylor et al. and entitled THERMAL SYSTEM WITH OVERSHOOT REDUCTION, the complete disclosure of which is incorporated herein by reference. In other embodiments, bypass valve 68 may be a pressure operated valve that allows fluid to flow along bypass line 64 if the fluid pressure in circulation channel 36 exceeds the cracking pressure of the bypass valve 68. Still further, in some embodiments, bypass valve 68 may be omitted and fluid may be allowed to flow through both bypass line 64 and into outlet manifold 54.

The incoming fluid flowing into inlet manifold 78 from inlet ports 62 and/or bypass line 64 travels back toward pump 34 and into an air remover 70. Air remover 70 includes any structure in which the flow of fluid slows down sufficiently to allow air bubbles contained within the circulating fluid to float upwardly and escape to the ambient surroundings. In some embodiments, air remover 70 is constructed in accordance with any of the configurations disclosed in commonly assigned U.S. patent application Ser. No. 15/646,847 filed Jul. 11, 2017, by inventor Gregory S. Taylor and entitled THERMAL CONTROL SYSTEM, the complete disclosure of which is hereby incorporated herein by reference. After passing through air remover 70, the circulating fluid flows past a valve 72 positioned beneath fluid reservoir 32. Fluid reservoir 32 supplies fluid to thermal control unit 22 and circulation channel 36 via valve 72, which may be a conventional check valve, or other type of valve, that automatically opens when reservoir 32 is coupled to thermal control unit 22 and that automatically closes when reservoir 32 is decoupled from thermal control unit 22 (see FIG. 2). After passing by valve 72, the circulating fluid travels to pump 34 and the circuit is repeated.

Controller 60 of thermal control unit 22 is contained within main body 30 of thermal control unit 22 and is in electrical communication with pump 34, heat exchanger 40, outlet temperature sensor 56, bypass valve 68, a patient sensor module 74, user interface 76, a memory 80, and, in some embodiments, a location sensor 92. Controller 60 includes any and all electrical circuitry and components necessary to carry out the functions and algorithms described herein, as would be known to one of ordinary skill in the art. Generally speaking, controller 60 may include one or more microcontrollers, microprocessors, and/or other programmable electronics that are programmed to carry out the functions described herein. It will be understood that controller 60 may also include other electronic components that are programmed to carry out the functions described herein, or that support the microcontrollers, microprocessors, and/or other electronics. The other electronic components include, but are not limited to, one or more field programmable gate arrays, systems on a chip, volatile or nonvolatile memory, discrete circuitry, integrated circuits, application specific integrated circuits (ASICs) and/or other hardware, software, or firmware, as would be known to one of ordinary skill in the art. Such components can be physically configured in any suitable manner, such as by mounting them to one or more circuit boards, or arranging them in other manners, whether combined into a single unit or distributed across multiple units. Such components may be physically distributed in different positions in thermal control unit 22, or they may reside in a common location within thermal control unit 22. When physically distributed, the components may communicate using any suitable serial or parallel communication protocol, such as, but not limited to, CAN, LIN, Firewire, I-squared-C, RS-232, RS-465, universal serial bus (USB), etc.

User interface 76, which may be implemented as a control panel or in other manners, allows a user to operate thermal control unit 22. User interface 76 communicates with controller 60 and includes a display 88 and a plurality of dedicated controls 82a, 82b, 82c, etc. Display 88 may be implemented as a touch screen, or, in some embodiments, as a non-touch-sensitive display. Dedicated controls 82 may be implemented as buttons, switches, dials, or other dedicated structures. In any of the embodiments, one or more of the functions carried out by a dedicated control 82 may be replaced or supplemented with a touch screen control that is activated when touched by a user. Alternatively, in any of the embodiments, one or more of the controls that are carried out via a touch screen can be replaced or supplemented with a dedicated control 82 that carries out the same function when activated by a user.

Through either dedicated controls 82 and/or a touch screen display (e.g. display 88), user interface 76 enables a user to turn thermal control unit 22 on and off, select a mode of operation, select a target temperature for the fluid delivered to thermal pads 24, select a patient target temperature, and control other aspects of thermal control unit 22, many of which are discussed in greater detail below. In some embodiments, user interface 76 may include a pause/event control, a medication control, and/or an automatic temperature adjustment control that operate in accordance with the pause event control 66b, medication control 66c, and automatic temperature adjustment control 66d disclosed in commonly assigned U.S. patent application Ser. No. 62/577,772 filed on Oct. 27, 2017, by inventors Gregory Taylor et al. and entitled THERMAL SYSTEM WITH MEDICATION INTERACTION, the complete disclosure of which is incorporated herein by reference. Such controls may be activated as touch screen controls or dedicated controls 82.

In those embodiments where user interface 76 allows a user to select from different modes for controlling the patient's temperature, the different modes include, but are not limited to, a manual mode and an automatic mode, both of which may be used for cooling and heating the patient. In the manual mode, a user selects a target temperature for the fluid that circulates within thermal control unit 22 and that is delivered to thermal pads 24. Thermal control unit 22 then makes adjustments to heat exchanger 40 in order to ensure that the temperature of the fluid exiting supply hoses 26a is at the user-selected temperature.

Another one of the modes is an automatic mode. When the user selects the automatic mode, the user selects a target patient temperature, rather than a target fluid temperature. After selecting the target patient temperature, controller 60 makes automatic adjustments to the temperature of the fluid in order to bring the patient's temperature to the desired patient target temperature. In this mode, the temperature of the circulating fluid may vary as necessary in order to bring about the target patient temperature.

In order to carry out the automatic mode, thermal control unit 22 utilizes patient sensor module 74. Patient sensor module 74 includes one or more patient temperature sensor ports 84 (FIGS. 2 & 3) that are adapted to receive one or more conventional patient temperature sensors or probes 86. The patient temperature sensors 86 may be any suitable patient temperature sensor that is able to sense the temperature of the patient at the location of the sensor. In one embodiment, the patient temperature sensors are conventional Y.S.I. 400 probes marketed by YSI Incorporated of Yellow Springs, Ohio, or probes that are YSI 400 compliant or otherwise marketed as 400 series probes. In other embodiments, different types of sensors may be used with thermal control unit 22. Regardless of the specific type of patient temperature sensor used in thermal control system 20, each temperature sensor 86 is connected to a patient temperature sensor port 84 positioned on thermal control unit 22. Patient temperature sensor ports 84 are in electrical communication with controller 60 and provide current temperature readings of the patient's temperature.

Controller 60, in some embodiments, controls the temperature of the circulating fluid using closed-loop feedback from temperature sensor 56. That is, controller 60 determines (or receives) a target temperature of the fluid, compares it to the measured temperature from sensor 56, and issues a command to heat exchanger 40 that seeks to decrease the difference between the desired fluid temperature and the measured fluid temperature. In some embodiments, the difference between the fluid target temperature and the measured fluid temperature is used as an error value that is input into a conventional Proportional, Integral, Derivative (PID) control loop. That is, controller 60 multiplies the fluid temperature error by a proportional constant, determines the derivative of the fluid temperature error over time and multiplies it by a derivative constant, and determines the integral of the fluid temperature error over time and multiplies it by an integral constant. The results of each product are summed together and converted to a heating/cooling command that is fed to heat exchanger 40 and tells heat exchanger 40 whether to heat and/or cool the circulating fluid and how much heating/cooling power to use.

When thermal control unit 22 is operating in the automatic mode, controller 60 may use a second closed-loop control loop that determines the difference between a patient target temperature and a measured patient temperature. The patient target temperature is input by a user of thermal control unit 22 using user interface 76. The measured patient temperature comes from a patient temperature sensor 86 coupled to one of patient temperature sensor ports 84 (FIG. 3). Controller 60 determines the difference between the patient target temperature and the measured patient temperature and, in some embodiments, uses the resulting patient temperature error value as an input into a conventional PID control loop. As part of the PID loop, controller 60 multiplies the patient temperature error by a proportional constant, multiplies a derivative of the patient temperature error over time by a derivative constant, and multiplies an integral of the patient temperature error over time by an integral constant. The three products are summed together and converted to a target fluid temperature value. The target fluid temperature value is then fed to the first control loop discussed above, which uses it to compute a fluid temperature error.

It will be understood by those skilled in the art that other types of control loops may be used. For example, controller 60 may utilize one or more PI loops, PD loops, and/or other types of control equations. In some embodiments, the coefficients used with the control loops may be varied by controller 60 depending upon the patient's temperature reaction to the thermal therapy, among other factors. One example of such dynamic control loop coefficients is disclosed in commonly assigned U.S. patent application Ser. No. 62/577,772 filed on Oct. 27, 2017, by inventors Gregory Taylor et al. and entitled THERMAL SYSTEM WITH MEDICATION INTERACTION, the complete disclosure of which is incorporated herein by reference.

Regardless of the specific control loop utilized, controller 60 implements the loop(s) multiple times a second in at least one embodiment, although it will be understood that this rate may be varied widely. After controller 60 has output a heat/cool command to heat exchanger 40, controller 60 takes another patient temperature reading (from sensor 86) and/or another fluid temperature reading (from sensor 56) and re-performs the loop(s). The specific loop(s) used, as noted previously, depends upon whether thermal control unit 22 is operating in the manual mode or automatic mode.

It will also be understood by those skilled in the art that the output of any control loop used by thermal control unit 22 may be limited such that the temperature of the fluid delivered to thermal pads 24 never strays outside of a predefined maximum and a predefined minimum. Examples of such a predefined maximum temperature and predefined minimum temperature are disclosed and discussed in greater detail in commonly assigned U.S. patent application Ser. No. 16/222,004 filed Dec. 17, 2018, by inventors Gregory S. Taylor et al. and entitled THERMAL SYSTEM WITH GRAPHICAL USER INTERFACE, the complete disclosure of which is incorporated herein by reference. The predefined minimum temperature is designed as a safety temperature and may be set to about four degrees Celsius, although other temperatures may be selected. The predefined maximum temperature is also implemented as a safety measure and may be set to about forty degrees Celsius, although other values may be selected.

In some embodiments of thermal control unit 22, such as the embodiment shown in FIG. 3, thermal control unit 22 also includes a reservoir valve 96 that is adapted to selectively move fluid reservoir 32 into and out of line with circulation channel 36. Reservoir valve 96 is positioned in circulation channel 36 between air remover 70 and valve 72, although it will be understood that reservoir valve 96 may be moved to different locations within circulation channel 36. Reservoir valve 96 is coupled to circulation channel 36 as well as a reservoir channel 98. When reservoir valve 96 is open, fluid from air remover 70 flows along circulation channel 36 to pump 34 without passing through reservoir 32 and without any fluid flowing along reservoir channel 98. When reservoir valve 96 is closed, fluid coming from air remover 70 flows along reservoir channel 98, which feeds the fluid into reservoir 32. Fluid inside of reservoir 32 then flows back into circulation channel 36 via valve 72. Once back in circulation channel 36, the fluid flows to pump 34 and is pumped to the rest of circulation channel 36 and thermal pads 24 and/or bypass line 64. In some embodiments, reservoir valve 96 is either fully open or fully closed, while in other embodiments, reservoir valve 96 may be partially open or partially closed. In either case, reservoir valve 96 is under the control of controller 60.

In those embodiments of thermal control unit 22 that include a reservoir valve, thermal control unit 22 may also include a reservoir temperature sensor 100. Reservoir temperature sensor 100 reports its temperature readings to controller 60. When reservoir valve 96 is open, the fluid inside of reservoir 32 stays inside of reservoir 32 (after the initial drainage of the amount of fluid needed to fill circulation channel 36 and thermal pads 24). This residual fluid is substantially not affected by the temperature changes made to the fluid within circulation channel 36 as long as reservoir valve 96 remains open. This is because the residual fluid that remains inside of reservoir 32 after circulation channel 36 and thermal pads 24 have been filled does not pass through heat exchanger 40 and remains substantially thermally isolated from the circulating fluid. Two results flow from this: first, heat exchanger 40 does not need to expend energy on changing the temperature of the residual fluid in reservoir 32, and second, the temperature of the circulating fluid in circulation channel 36 will deviate from the temperature of the residual fluid as the circulating fluid circulates through heat exchanger 40.

In some embodiments, controller 60 utilizes a temperature control algorithm to control reservoir valve 96 that, in some embodiments, is the same as the temperature control algorithm 160 disclosed in commonly assigned U.S. patent application Ser. No. 62/577,772 filed on Oct. 27, 2017, by inventors Gregory Taylor et al. and entitled THERMAL SYSTEM WITH MEDICATION INTERACTION, the complete disclosure of which is incorporated herein by reference. In other embodiments, controller 60 utilizes a different control algorithm. In still other embodiments, thermal control unit 22 is modified to omit reservoir valve 96, reservoir channel 98, and reservoir temperature sensor 100. Thermal control unit 22 may also be modified such that reservoir 32 is always in the path of circulation channel 36. Still other modifications are possible.

It will be understood that the particular order of the components along circulation channel 36 of thermal control unit 22 may be varied from what is shown in FIG. 3. For example, although FIG. 3 depicts pump 34 as being upstream of heat exchanger 40 and air separator 70 as being upstream of pump 34, this order may be changed. Air separator 70, pump 34, heat exchanger 40 and reservoir 32 may be positioned at any suitable location along circulation channel 36. Indeed, in some embodiments, reservoir 32 is moved so as to be in line with and part of circulation channel 36, rather than external to circulation channel 36 as shown in FIG. 3, thereby forcing the circulating fluid to flow through reservoir 32 rather than around reservoir 32. Further details regarding the construction and operation of one embodiment of thermal control unit 22 that are not described herein may be found in commonly assigned U.S. patent application Ser. No. 14/282,383 filed May 20, 2014, by inventors Christopher Hopper et al. and entitled THERMAL CONTROL SYSTEM, the complete disclosure of which is incorporated herein by reference.

In some embodiments, thermal pads 24 are constructed in accordance with any of the thermal pads disclosed in any of the following commonly assigned U.S. patent applications: Ser. No. 15/675,061 filed Aug. 11, 2017, by inventors James Galer et al. and entitled THERMAL THERAPY DEVICES; Ser. No. 62/778,034 filed Dec. 11, 2018, by inventors Andrew M. Bentz et al. and entitled THERMAL SYSTEM WITH THERMAL PAD FILTERS; and Ser. No. 15/675,066 filed Aug. 11, 2017, by inventor James K. Galer and entitled THERMAL SYSTEM, the complete disclosures of all of which are incorporated herein by reference. Still other types of thermal pads 24 may be used with thermal control system 20, and thermal control unit 22 may be modified from its construction described herein in order to accommodate the particular thermal therapy pad(s) it is used with.

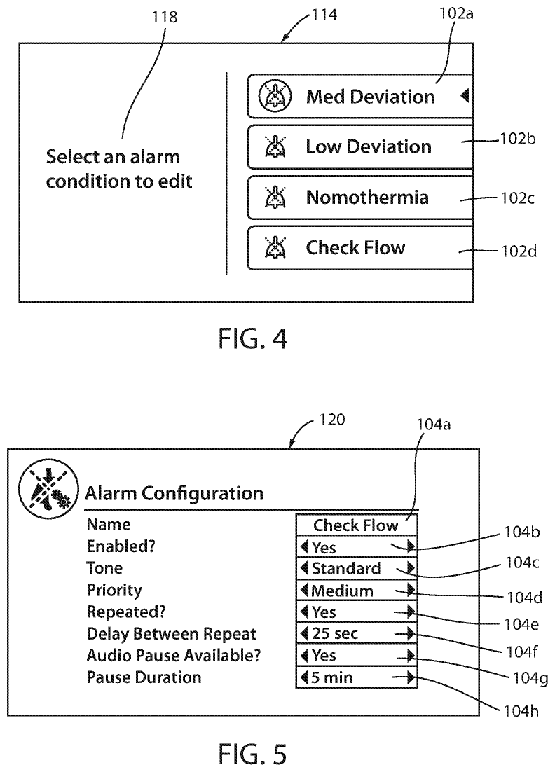

Memory 80 (FIG. 3) may be any type of conventional non-volatile memory, such as, but not limited to flash memory, one or more hard drives, one or more EEPROMs, etc. Memory 80 may also be implemented to include more than one of these types of memories in combination. In the embodiment shown in FIG. 3, memory 80 of thermal control unit 22 includes a plurality of items stored therein, such as one or more sets of each of the following: alarm conditions 102, alarm characteristics 104, therapy profiles 106, user data 108, location data 110, and auxiliary sensor data 112. These items are able to entered into memory 80 locally via user interface 76. Additionally, in some embodiments, any of these items in memory 80 may be transferred (wired or wirelessly) to thermal control unit 22 from another device, such as, but not limited to, a server, another thermal control unit, a flash drive, etc. Memory 80 and the items stored therein are discussed in greater detail below with respect to FIGS. 4-12.

FIG. 4 illustrates one example of an alarm condition selection screen 114 that is displayable on display 88 of thermal control unit 22. Alarm condition selection screen 114 is displayable on display 88 by controller 60 when a user wishes to customize one or more aspects of the alarms that are issued by thermal control unit 22. Alarm condition selection screen 114 includes a plurality of alarm conditions 102a-d and a message 118 instructing the user to select one of the alarm conditions 102a-d. Once the user selects a particular alarm condition 102, controller 60 is configured to display an alarm customization screen, such as alarm customization screen 120 of FIG. 5, that displays characteristics of the selected alarm condition 102 and that allows the user to customize those alarm characteristics, as will be discussed in more detail below.

Each of the alarm conditions 102 shown in FIG. 4 defines when controller 60 will issue an alarm. Although FIG. 4 only shows four such alarm conditions 102a-d, it will be understood that controller 60 is configured to issue more than just these four alarms. In at least one embodiment, memory 80 includes a default set of alarm conditions 102 that instruct controller 60 to issue an alarm when any one of the following conditions occur: (a) one or more of the patient temperature sensors 86 malfunctions; (b) one or more of the patient temperature sensors 86 is disconnected from its corresponding port 84; (c) the patient's temperature deviates outside of a first range (e.g. a narrow range); (d) the patient's temperature deviates outside of a second range (e.g. a wider range than the first range); (e) the patient's temperature devices from the normal human body temperature (37° C.) by more than a threshold; (f) the temperature of the fluid delivered to the outlet ports 58 deviates outside of an acceptable range; (g) a sensor (not shown) detects that there is insufficient fluid inside thermal control unit 22; (h) a flow sensor (not shown) detects that less than an acceptable amount of fluid is being pumped through or out of thermal control unit 22 (e.g. out of outlet manifold 54); (i) a user pauses a therapy session (via user interface 76); and (j) a battery included within thermal control unit 22 discharges below a threshold level. In such embodiments, controller 60 is configured to display all of these alarm conditions 102 on alarm selection screen 114 (or alternatively it is configured to display multiple alarm selection screens 114 that collectively include all of these alarm conditions 102).

Controller 60 monitors each of the alarm conditions 102 during operation of thermal control unit 22 and issues a corresponding alarm if it detects the occurrence of the alarm condition. Thus, for example, controller 60 monitors signals from patient temperature sensor 86 during operation of thermal control unit 22, and if those signals go outside of an expected range, or otherwise behave in a manner that is not expected, it concludes that the patient temperature sensor 86 is malfunctioning, and therefore issues an alarm corresponding to this condition. Similarly, controller 60 monitors one or more sensors (not shown) that detect the connection/disconnection of patient temperature sensor 86 to patient temperature probe port 84 and, if the sensor 86 is unplugged from the port 84, it issues the alarm corresponding to this condition. It can thus be seen that controller 60 monitors all of the corresponding conditions specified by alarm conditions 102 during operation of thermal control unit 22 and issues an alarm if it detects the presence and/or occurrence of one or more of these conditions.

Controller 60 is also configured to allow a user to add one or more additional alarm conditions to the default set of alarm conditions, as well as to remove one more alarm conditions from this default set of alarm conditions. One manner in which controller 60 is configured to allow a user to make these types of modifications is via alarm customization screen 120. Controller 60 is configured to display alarm customization screen 120 in response to a user touching (or otherwise selecting) one of alarm conditions 102a-d shown in alarm selection screen 114 (FIG. 4). In the particular example illustrated in FIGS. 4 and 5, the user has touched check flow alarm condition 102 in FIG. 4 and controller 60 has displayed an alarm customization screen 120 in FIG. 5 that corresponds to the check flow alarm condition 102. If the user were to select medium deviation condition alarm 102a from screen 114, controller 60 is configured to display an alarm customization screen 120 that is specific to the medium deviation alarm condition 102a. Likewise, if the user were to select low deviation alarm condition 102b from screen 114, controller 60 is configured to display an alarm customization screen 120 that is specific to the low deviation alarm condition 102b. Similarly, if the user were to select normothermia alarm condition 102c, controller 60 is configured to display an alarm customization screen 120 that is specific to the normothermia alarm condition 102c. Finally, if alarm selection screen 114 were to include additional, or different, alarm conditions 102, controller 60 is configured to display corresponding alarm customization screens 120 that are specific to each individual alarm condition 102.

Each alarm customization screen 120 that controller 60 is configured to display includes a list of characteristics of the alarm for the corresponding alarm condition 102. For example, as shown in FIG. 5, alarm customization screen 120 includes eight alarm characteristics 104a. It will be appreciated that not only may this number of characteristics 104 be varied, but that the specific content of any one or more of these characteristics may also or alternatively be varied. In the example shown in FIG. 5, controller 60 displays the following eight characteristics of the check flow alarm condition 102d: (1) the name 104a of the alarm condition; (2) the enablement/disablement state 104b of the alarm condition; (3) the tone 104c of the alarm that is issued in response to detecting the alarm condition; (4) the priority 104d of the alarm; (5) the repeat status 104e of the alarm; (6) a delay amount 104f between repetitions of the alarm; (7) an audio pause availability status 104g of the alarm; and (8) a pause duration 104h.

In one embodiment, controller 60 is configured to list these same alarm characteristics 104 on each of the customization screens 120 corresponding to each one of the alarm conditions 102. In other embodiments, individual alarm conditions 102 may have different sets of characteristics 104 associated with them. Regardless of the specific number of alarm characteristics 104 shown on a customization screen 120, or the specific choice of alarm characteristics 104 that are displayed on a customization screen 120, controller 60 is configured to allow a user to modify each of the alarm characteristics 104. Such modification takes place by touching, or otherwise selecting, the alarm characteristic 104 that is desired to be changed.

For example, if the user wishes to change the name of an alarm condition 102, he or she touches the alarm name characteristic 104a on screen 120 (FIG. 5). In one embodiment, when alarm name characteristic 104a is touched, controller 60 is configured to display an alphanumeric keyboard popup on display 88 that allows the user to type in a different name for the alarm condition. Once entered, controller 60 ceases to display the keyboard popup and controller 60 saves the new name entered by the user. Such a name change will affect the name displayed by controller 60 on alarm selection screen 114 for the corresponding alarm condition 102. The user is able to change any of the other alarm characteristics 104 in a similar manner; that is, by touching the characteristic 104 desired to be changed and then using the arrows positioned adjacent that characteristic 104 to change the value or setting for that particular characteristics 104.

If the user wishes to disable a particular alarm condition 102, he or she touches one of the arrows adjacent the “enabled” alarm characteristic 104b until the word “no” is displayed. As a result of disabling the alarm condition 102, controller 60 does not issue an alarm when that corresponding condition is detected. Thus, in the example of FIG. 5, if the check flow alarm condition 102d were disabled, controller 60 would not issue an alarm if the flow rate of the fluid within circulation channel 36 (and/or delivered to thermal pads 24) fell below the threshold that is monitored by controller 60 and otherwise used to trigger this alarm.

If the user wishes to change the tone of the sound emitted by thermal control unit 22 (such by a speaker, a beeper, a buzzer, or other sound-generating device incorporated therein), he or she touches one of the arrows adjacent the “tone” alarm characteristic 104c (FIG. 5). Touching these arrows causes controller 60 to scroll through the different options for the tone that is emitted when this alarm condition 102 is detected. The particular options for the “tone” characteristic may vary from thermal control unit to thermal control unit, but generally include options for at least one of the pitch, strength, quality, and/or timbre of the emitted alarm sound.

Controller 60 also enables the user to change the priority of the alarm issued for each alarm condition 102. To make such a change, the user touches one of the arrows adjacent the “priority” characteristic 104d (FIG. 5). Touching these arrows causes controller 60 to scroll through the different options for the priority, such as, but not limited to, a “high,” “medium,” and “low” priority. In one embodiment, controller 60 is configured to respond to a change in the “priority” characteristic 104d by changing the alarm in the manner set forth in the International Electrotechnical Commission (IEC) 60601-1-8 standard (“Audible Alarms in Medical Equipment”). In other embodiments, controller 60 may adjust the alarm priority in accordance with other standards and/or in other manners.

Controller 60 is further configured to allow the user to change whether any of the alarms issued for any of alarm conditions 102 are repeated or not. To make such a change, the user selects one of the arrows positioned adjacent the “repeated” alarm characteristic 104e (FIG. 5). Touching one of these arrows causes controller 60 to toggle between displaying a “yes” and a “no.” By selecting “no,” controller 60 will not repeat the corresponding alarm, but instead will issue it only once in response to detecting the corresponding alarm condition 102.

If the user chooses to have an alarm repeated, controller 60 allows the user to select how much time controller 60 waits between repetitions of the alarm. The user makes this choice by selecting one of the arrows positioned adjacent the “delay between repeat” alarm characteristic 104f. Touching the adjacent left arrow reduces the time period, while touching the adjacent right arrow increase the time period. Once the desired time period is selected, controller 60 uses the selected value as the delay period between repeated issuance of that particular alarm.

Controller 60 is also configured to allow the user to change whether any of the alarms issued for any of the alarm conditions 102 can be paused by a user. In one embodiment, when an alarm is issued, controller 60 displays a pause icon (not shown) on display 88 that, when touched by a user, temporarily pauses the emitted alarm sound. In another embodiment, user interface 76 includes a dedicated control 82 that, when pressed or otherwise activated, temporarily pauses the emitted alarm sound. Regardless of the specific manner in which the pause control is implemented, if the user does not wish to be able to pause a particular alarm, he or she can disable the ability of the user to pause an alarm by changing the “audio pause available” characteristic 104g (FIG. 5). Pressing on one of the arrows adjacent to this characteristic causes controller 60 to toggle between displaying a “yes” and a “no.” When the “no” is selected, controller 60 does not allow a user to pause that particular alarm. Consequently, in those embodiments in which a pause icon is displayed on display 88, controller 60 either does not display the pause icon when the corresponding alarm is issued, or it disables the pause icon when the corresponding alarm is issued. In those embodiments in which the pause control is a dedicated control 82, controller disables that control 82 for the corresponding alarm.

If the user chooses to allow a particular alarm to be paused, controller 60 is configured to also allow the user to customize how long the alert is paused for. The user selects this pause time by touching one of the arrows positioned adjacent the “pause duration” characteristic 104h (FIG. 5). Controller 60 responds to the touching of these arrows by either decreasing the pause time (e.g. left arrow) or increasing the pause time (e.g. right arrow). Once the desired pause time value is selected, controller 60 thereafter uses the selected time value when pausing the corresponding alarm. That is, when the user presses the pause control, controller 60 stops the audible portion of the alarm for the length of time specified by characteristic 104h, and upon expiration of that time period, resumes the audible portion of the alarm (if the condition triggering the alarm has not yet been remedied).

As was noted, the particular alarm characteristics 104 shown in FIG. 5 are but one example of the types of alarm characteristics that may be customizable by a user of thermal control unit 22. In other embodiments, additional or fewer alarm characteristics 104 may be customizable, and/or different characteristics from the specific characteristics 104 shown in FIG. 5 may be customizable.

In some embodiments, the ability of a user to customize the alarm conditions 102 and/or alarm characteristics 104 is restricted to only authorized personnel. In such embodiments, controller 60 may be configured to only allow users who enter a valid password to change the alarm settings (i.e. conditions and/or characteristics). In other embodiments, other manners of restricting access to the alarm customization features of thermal control unit 22 may be implemented, such as, but not limited to, facial recognition, fingerprint (or other biometric) recognition, etc. By restricting access to the customization features of thermal control unit 22 to only authorized personnel, the actual users of thermal control unit 22 during a therapy session may be prevented from making changes to the alarm settings. Administrators of a healthcare facility can therefore dictate what types of alarms are to be utilized, as well as their characteristics, and the nurse, doctors, and other personnel who actually use the thermal control unit 22 to treat a patient may be prevented from changing these alarm settings. It will therefore be understood that the use of the term “user” herein encompasses not only the individuals who utilize thermal control unit 22 to control a person's temperature (e.g. doctors, nurses, etc.), but also users who configure the settings of thermal control unit 22 prior to, or after, individual therapy sessions (e.g. administrators).

It can be seen that thermal control unit 22 permits a large amount of alarm customization to be implemented. For example, if thermal control unit 22 includes ten default alarm conditions 102, and each one of those alarm conditions includes ten alarm characteristics 104 that can be modified, such a thermal control unit would include one hundred individual alarm characteristics 104 that could be customized. Because of such large numbers, and in order to reduce the workload of users of thermal control unit 22, controller 60 is configured in some embodiments to store one or more defined sets alarm conditions 102 and their respective characteristics 104. The user of thermal control unit 22 can then select one of these defined sets of alarms conditions 102 and characteristics 104, and controller 60 automatically implements the selected set.

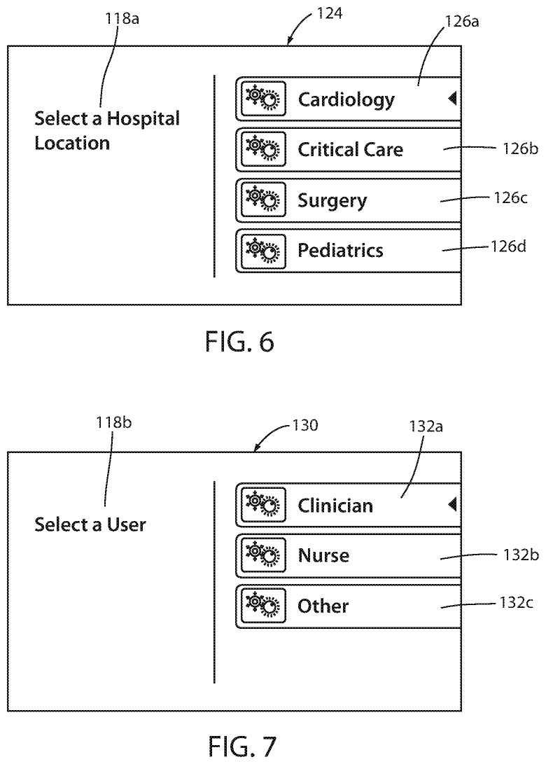

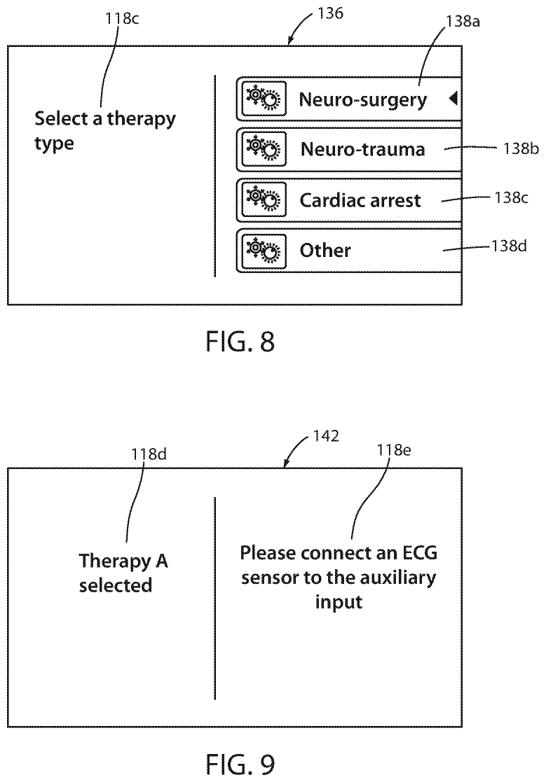

The defined sets of alarm conditions 102 and alarm characteristics 104 may be classified in different manners. For example, in one embodiment, thermal control unit 22 includes sets of alarm conditions 102 and alarm characteristics 104 that are classified according to particular locations within a healthcare facility. The classification of the sets of alarm conditions/characteristics in this manner is discussed in more detail below with respect to FIG. 6. In another embodiment, thermal control unit 22 includes sets of alarm conditions 102 and alarm characteristics 104 that are classified according to particular users of thermal control unit 22, and the classification of the sets of alarm conditions/characteristics in this manner is discussed in more detail below with respect to FIG. 7. In still another embodiment, thermal control unit 22 includes sets of alarm conditions 102 and alarm characteristics 104 that are classified according to types of thermal treatments, and the classification of the sets of alarm conditions/characteristics in this manner is discussed in more detail below with respect to FIG. 8. Still further, in some embodiments, thermal control unit 22 is configured to include sets of alarm conditions 102 and alarm characteristics 104 that are classified in multiple manners, such as combinations of locations, users, therapy types, and/or other classifications.

FIG. 6 illustrates an example of a location selection screen 124 that is displayed by controller 60 on display 88 in those embodiments of thermal control unit 22 that include defined sets of alarm conditions 102 and/or alarm characteristics 104 that are classified according to location. Location selection screen 124 includes a message 118a instructing the user to select a location. Location selection screen 124 also includes a listing of locations 126a-d. Each location 126 corresponds to a particular location within the hospital, or other healthcare facility, in which thermal control unit 22 is used. Although FIG. 6 shows four specific locations 126, it will be understood that thermal control unit 22 may include more than, or less than, four locations, and that the specific locations identified in FIG. 6 may be varied. In the particular example of FIG. 6, location 126a corresponds to the cardiology department of the healthcare facility; location 126b corresponds to the critical care department of the healthcare facility; location 126c corresponds to the surgical department of the healthcare facility; and location 126d corresponds to the pediatrics department of the healthcare facility.

When a user of thermal control unit 22 selects one of locations 126a-d, controller 60 is configured to implement the set of alarm conditions 102 and alarm characteristics 104 that correspond to the particular location selected by the user. Thus, the different departments of the healthcare facility may decide to customize the alarms of thermal control unit 22 differently for when thermal control unit 22 is used in their department. In this manner, for example, the cardiology department may choose to omit issuing an alarm when the battery is discharged below a certain state and to issue only a low priority alarm for when the patient's temperature deviates outside of a small range, while the surgery department may choose to utilize the low battery alarm and to issue a high priority alarm when the patient's temperature deviates outside of the small range. As another example, the cardiology department might utilize twelve alarm conditions 102, and customize the characteristics 104 of eight of those in the same manner, and individually customize the characteristics 104 of the remaining four in different manners, while the pediatrics department might utilize fourteen alarm conditions 102, twelve of which have their characteristics 104 customized in the same manner and two of which have their characteristics 104 customized in different manners from the other alarm conditions. These are, of course, just two types of customizations out of thousands of different manners in which the alarms of thermal control unit 22 can be customized according to different locations/departments within a healthcare facility.

In some embodiments of thermal control unit 22, thermal control unit 22 includes a location sensor 92 that automatically detects the location of thermal control unit 22 within a healthcare facility. In such embodiments, once a set of alarm conditions/characteristics for a particular location have been input and saved in memory 80, thermal control unit 22 uses location sensor 92 to automatically detect its location within the healthcare facility and then automatically implements the corresponding set of alarm conditions/characteristics for that location. In such embodiments, the user does not need to manually enter the location of the thermal control unit 22.

In those embodiments of thermal control unit 22 that include one or more location sensors 92, such location sensors 92 may take on a variety of different forms. For example, in one embodiment, thermal control unit 22 includes a WiFi transceiver that communicates with the healthcare facility's local area network via the network's wireless access points, and controller 60 determines its location relative to the known locations of these access points based upon the detected signal strengths from these access points. In another example, thermal control unit 22 may determine its location using any of the same methods and/or sensors disclosed for determining patient support apparatus location in commonly assigned U.S. Pat. No. 9,838,836 issued Dec. 5, 2017, to inventors Michael J. Hayes et al. and entitled PATIENT SUPPORT APPARATUS COMMUNICATION SYSTEMS, the complete disclosure of which is incorporated herein by reference. Still other automatic location detection methods may be used, including, but not limited to, the use of cellular network trilateration and/or Global Positioning System (GPS) sensors.

The sets of alarm conditions 102 and alarm characteristics 104 that correspond to the different locations within a healthcare facility are input into memory 80 by authorized users of thermal control unit 22. In some embodiments, the user needs to enter a password or other credentials in order to define these sets. Further, in some embodiments, the user needs to go through the process defined above with respect to FIGS. 4 and 5 to define the individual alarm conditions 102 and alarm characteristics 104 for each location. Once this process is completed, the user is able to assign a location name (e.g. cardiology) to the set of alarm conditions 102 and characteristics 104 that he or she has just defined. After the name is assigned, controller 60 is configured to display that location and its name on location selection screen 124, and, if selected by the subsequent user, to implement the set of alarm conditions 102 and characteristics 104 that were input during that process.

In at least some modified embodiments, thermal control unit 22 is configured to accept predefined sets of alarm conditions and alarm characteristics from one or more external devices, such as, but not limited to, a server on a network, a portable computer, a thumb (flash) drive, or another device. In these modified embodiments, the user does not necessarily need to manually go through the process described above with respect to FIGS. 4 and 5 to input sets of alarms conditions and characteristics that correspond to locations. In some of these modified embodiments, the transfer of the predefined sets of alarm conditions and characteristics classified according to locations may be carried out in any of the manners of transferring data to and/or from a thermal control unit that are disclosed in commonly assigned U.S. patent application Ser. No. 15/616,574 filed Jun. 7, 2017, by inventors Gregory Taylor et al. and entitled THERMAL CONTROL SYSTEM, the complete disclosure of which is incorporated herein by reference. Still other manners of transferring predefined sets of alarm conditions and alarm characteristics to memory 80 of thermal control unit 22 may also, or alternatively, be used.

FIG. 7 illustrates an example of a user selection screen 130 that is displayed by controller 60 on display 88 in those embodiments of thermal control unit 22 that include defined sets of alarm conditions 102 and/or alarm characteristics 104 that are classified according to user. User selection screen 130 includes a message 118b instructing the user to select a class of user. User selection screen 130 also includes a listing of users 132a-c defined according to the class of the users who may be using thermal control unit 22. Although FIG. 7 shows three classes of users 132a-c, it will be understood that thermal control unit 22 may include more than, or less than, three classes of users, and that the specific classes identified in FIG. 7 may be varied. In the particular example of FIG. 7, user class 132a corresponds to clinicians; user class 132b corresponds to nurses; and class 132c corresponds to other types of users.

When a user of thermal control unit 22 selects one of the users 132a-c, controller 60 is configured to implement the set of alarm conditions 102 and alarm characteristics 104 that correspond to that particular class of user. Thus, the users of thermal control unit 22 are able to have the alarms of thermal control unit 22 customized according to their specific preferences. In this manner, for example, nurses might choose to issue an alarm when the battery is discharged below a certain state and to issue a high priority alarm for when the patient's temperature deviates outside of a small range, while clinicians might choose to omit the low battery alarm and to issue a low priority alarm when the patient's temperature deviates outside of the small range. This is, of course, just one type of customization out of thousands of different manners in which the alarms of thermal control unit 22 can be customized according to users.

In some embodiments of thermal control unit 22, thermal control unit 22 includes a user sensor (not shown) that automatically detects the type of user of thermal control unit 22. In such embodiments, once a set of alarm conditions/characteristics for a particular user have been input and saved in memory 80, thermal control unit 22 uses the user sensor to automatically detect the current type of user and then automatically implement the corresponding set of alarm conditions/characteristics for that particular user. In such embodiments, the user does not need to manually identify himself or herself (or manually identify the class of users to which they belong).

In those embodiments of thermal control unit 22 that include one or more user sensors, such user sensors may take on a variety of different forms. For example, in one embodiment, thermal control unit 22 includes an RF ID sensor that is adapted to detect RF ID badges worn by healthcare personnel. In such situations, the ID contained within each badge either contains an identification of the type of user (e.g. nurse, clinician, etc.), or thermal control unit 22 includes a database of user IDs along with a table, or other data structure, that correlates each ID to a particular class of user. Based on the detected ID and corresponding user class, controller 60 selects the appropriate set of alarm conditions and characteristics.

In an another alternative embodiment, thermal control unit 22 includes one or more near field sensors that are adapted to detect near field badges, cards, or other objects having a near field transceiver integrated into them. Users of thermal control unit 22 carry the near field badges, cards, or other objects with them and pass them within proximity to the near field sensor onboard thermal control unit 22 when they approach thermal control unit 22. In response to detecting the badge, card, or other near field object, controller 60 automatically determines the user class and selects the appropriate set of alarm conditions and characteristics.

In still another embodiment, thermal control unit 22 includes one or more cameras, or other images sensors, that are adapted to capture one or more images of the user while he or she is using thermal control unit 22. Based on the captured images, controller 60 executes facial recognition software to determine who the user is. Once the identity of the user is determined, controller 60 determines what class of user that particular individual corresponds to and selects the appropriate set of alarm conditions and characteristics. In this embodiment, thermal control unit 22 may include a network transceiver (e.g. Ethernet, WiFi, etc.) that communicates with a local area network of a healthcare facility and accesses photographs of known authorized individuals. Alternatively, thermal control unit 22 may include a port (e.g. USB, Ethernet, etc.) for enabling a user to upload data defining the faces of all of the authorized users of thermal control unit 22, as well as the class of user corresponding to the facial data of each user, thereby providing controller 60 with the necessary data to carry out the facial recognition process.

Regardless of what type of user sensor(s) (if any) that thermal control unit 22 includes, controller 60 is configured in some embodiments to classify the sets of alarm conditions 102 and alarm characteristics 104 according to individual users, rather than user classes. Thus, in such embodiments, instead of controller 60 displaying “clinician” user class 132a and “nurse” class 132b on user selection screen 130 (FIG. 7), controller 60 displays the names of individual users, such as “Nurse A. Smith” or “Clinician J. Johnson” on screen 130. After the user selects one of these individuals, controller 60 then implements a set of alarm conditions 102 and alarm characteristics 104 that are personalized to that particular user.

In at least one embodiment, controller 60 is configured to allow a user to select multiple users (either by user class and/or by individual users) at the same time. In this embodiment, controller 60 is configured to communicate at least one of the sets of alarms remotely (corresponding to a first one of the users), while controller 60 issues the other set of alarms locally (i.e. aurally or visually at thermal control unit 22) and, in some cases, also remotely. For example, in this embodiment, controller 60 may be configured to send alarm messages to a clinician via text, email, instant messaging, paging, and/or a phone call when an alarm condition 102 corresponding to the set of alarm conditions 102 for that clinician is detected. If the alarm message sent to the clinician corresponds to an alarm condition 102 that is not on the set of alarm conditions for the other user, controller 60 does nothing additional regarding that particular alarm condition 102. However, if that particular alarm condition is part of the set of alarm conditions 102 for the other user, then controller 60 also takes the action specified in the corresponding alarm characteristics 104 for that other user. Thus, for example, if thermal control unit 22 detects that the patient's temperature has moved outside of a predefined range, controller 60 may issue an alarm in which it sends a message to the clinician (according to the clinician's set of alarm conditions and characteristics) and in which it emits a sound (according to the nurse's set of alarm conditions and characteristics). As another alternative, controller 60 may emit an alarm sound locally and send messages to both the nurse and the clinician. Still other variations are, of course, possible.

When thermal control unit 22 is configured to send messages to one or more individuals in response to an alarm condition being detected, thermal control unit 22 may include a network transceiver (e.g. a WiFi transceiver, an Ethernet transceiver, etc.) that couples thermal control unit 22 to the healthcare facility's local area network. Once coupled to this network, controller 60 may be configured to send the alarm message in any conventional manner, including, but not limited to, sending the message to one or more servers on the local area network that then forward the message to the appropriate mobile electronic device (e.g. smart phone, tablet, pager, laptop computer, etc.) of the corresponding nurse, clinician, or other user. Such servers include, but are not limited to, one or more commercially available paging, texting, emailing, and/or messaging servers.

It will be understood that the sets of alarm conditions 102 and alarm characteristics 104 that are defined according to users (whether individual users or user classes) may be separate and independent from the sets of alarm conditions 102 and 104 discussed above that are defined according to location, or they may alternatively be combined together, depending upon the particular embodiment of thermal control unit 22. With respect to the former embodiment, thermal control unit 22 may be configured to display both location selection screen 124 and user selection screen 130, and controller 60 implements whichever set of alarm conditions 102 and characteristics 104 was most recently selected by the user. For example, if the user selects a location 126b, controller 60 implements the set of alarm conditions and characteristics for that location 126b. However, if the user subsequently selects a user 132a, controller 60 stops using the set of alarm conditions and characteristics for location 126b and instead switches to the set of alarm conditions 102 and characteristics 104 that correspond to user 132a.

With respect to the latter embodiment, thermal control unit 22 may be configured to store a customized set of alarm conditions 102 and characteristics 104 for each user at each location. Thus, for example, if the user selects location 126b and does not select a particular user, controller 60 implements the set of alarm conditions and characteristics corresponding to location 126b. However, if the user selects location 126b and then subsequently selects user 132a, controller 60 implements a set of alarm conditions and characteristics that is specific to user 132a for that particular location 126b. This set of alarm conditions and characteristics may or may not be the same for user 132a at location 126a (or location 126c, etc.).

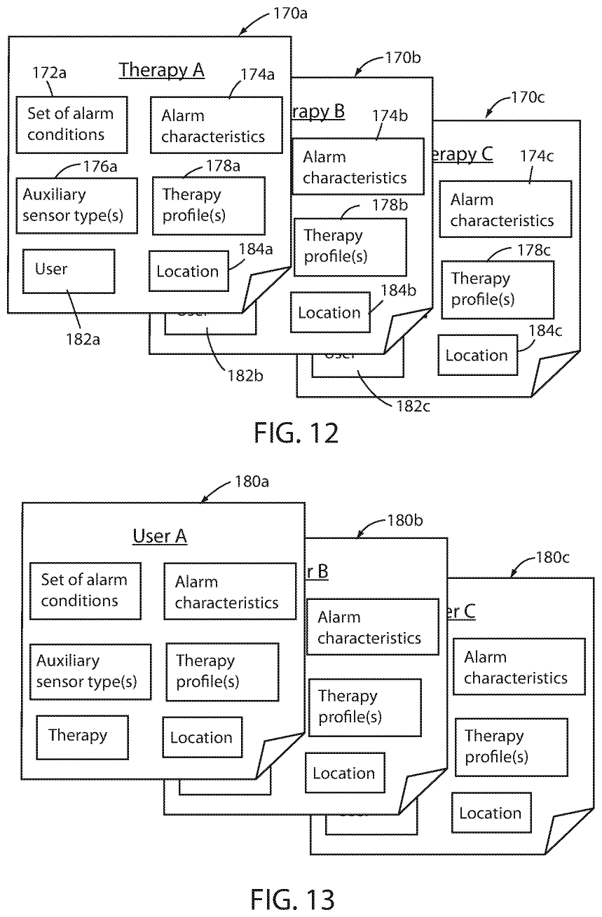

Regardless of whether or not thermal control unit 22 is configured to separately maintain, or to combine, the user sets and the location sets of alarm conditions and characteristics, controller 60 stores the contents of these sets in memory 80. As will be discussed in greater detail below, these sets may be combined with other customization data and stored in one or more records in memory 80, such as the records shown and discussed below with respect to FIGS. 12 and 13.

As with the sets of alarm conditions 102 and characteristics 104 that are defined according to location, the sets of alarm conditions 102 and characteristics 104 that are defined with respect to users may be input into memory 80 by authorized users of thermal control unit 22 utilizing user interface 76. In some embodiments, the user needs to enter a password or other credentials in order to define these sets. Additionally, the user may need to go through the process defined above with respect to FIGS. 4 and 5 to define the individual alarm conditions 102 and alarm characteristics 104 for each user. Once this process is completed, the user is able to assign a user name (e.g. nurse, Nurse A. Smith, etc.) to the set of alarm conditions 102 and characteristics 104 that he or she has just defined. After the name is assigned, controller 60 is configured to display that user on user selection screen 130, and, if selected by the subsequent user, to implement the set of alarm conditions 102 and characteristics that were input during that process.

In at least some modified embodiments, thermal control unit 22 is also, or alternatively, configured to accept predefined sets of alarm conditions and alarm characteristics from one or more external devices, such as, but not limited to, a server on a network, a portable computer, a thumb (flash) drive, or another device. In these modified embodiments, the user does not necessarily need to manually go through the process described above with respect to FIGS. 4 and 5 to input sets of alarms conditions and characteristics that correspond to users.

FIG. 8 illustrates an example of a therapy selection screen 136 that is displayed by controller 60 on display 88 in those embodiments of thermal control unit 22 that include defined sets of alarm conditions 102 and/or alarm characteristics 104 that are classified according to therapy types. Therapy selection screen 136 includes a message 118c instructing the user to select a type of therapy for which thermal control unit 22 will be used. Therapy selection screen 136 also includes a listing of different types of therapies 138a-d. Although FIG. 8 shows four types of therapy 138a-d, it will be understood that thermal control unit 22 may include more than, or less than, four types of therapy, and that the specific therapies identified in FIG. 8 may be varied. In the particular example of FIG. 8, controller 60 displays a neuro-surgery therapy 138a, a neuro-trauma therapy 138b, a cardiac arrest therapy 138c, and an “other” therapy class 138d.

When a user of thermal control unit 22 selects one of the therapies 183a-d, controller 60 is configured to implement the set of alarm conditions 102 and alarm characteristics 104 that correspond to that particular therapy. Thus, the users of thermal control unit 22 can have the alarms of thermal control unit 22 customized according to the different therapies for which thermal control unit 22 may be utilized. In this manner, for example, if thermal control unit 22 is being used to treat a patient after suffering a cardiac arrest, thermal control unit 22 might be configured to issue an alarm when the rate of change of the patient's temperature falls below a first threshold, or exceeds a second threshold, whereas if thermal control unit 22 is being used to treat a patient during neuro-surgery, thermal control unit 22 might be configured to issue an alarm if the patient's temperature deviates from a predetermined temperature by more than a threshold. Other manners of configuring the alarm conditions 102 and/or alarm characteristics 104 according to different therapies may, of course, also or alternatively be implemented.

The sets of alarm conditions 102 and characteristics 104 that are defined with respect to therapies 138 may be input into memory 80 by authorized users of thermal control unit 22 utilizing user interface 76. In some embodiments, the user needs to enter a password or other credentials in order to define these sets. Additionally, the user may need to go through the process defined above with respect to FIGS. 4 and 5 to define the individual alarm conditions 102 and alarm characteristics 104 for each type of therapy. Once this process is completed, the user is able to assign a therapy name (e.g. cardiac arrest) to the set of alarm conditions 102 and characteristics 104 that he or she has just defined. After the name is assigned, controller 60 is configured to display that therapy on therapy selection screen 136, and, if selected by the subsequent user, to implement the set of alarm conditions 102 and characteristics that were input during that process.

In at least some modified embodiments, thermal control unit 22 is also, or alternatively, configured to accept predefined sets of alarm conditions and alarm characteristics from one or more external devices, such as, but not limited to, a server on a network, a portable computer, a thumb (flash) drive, or another device. In these modified embodiments, the user does not necessarily need to manually go through the process described above with respect to FIGS. 4 and 5 to input sets of alarms conditions and characteristics that correspond to different therapies.

Further, just as the user-defined sets of alarm conditions 102 and alarm characteristics 104 may be implemented separately from, or in combination with, the location-defined sets of alarm conditions 102 and alarm characteristics, the therapy-defined sets of alarm conditions 102 and alarm characteristics 104 may be implemented separately from, or in combination with, one or both of the user-defined and/or location-defined sets of alarm conditions and characteristics. Thus, for example, thermal control unit 22 might include a set of alarm conditions and characteristics that is unique to, for example, the usage of thermal control unit 22 by Doctor J. Johnson when he or she is treating a cardiac arrest patient in the critical care department of the healthcare facility. Other examples, of course, are possible.

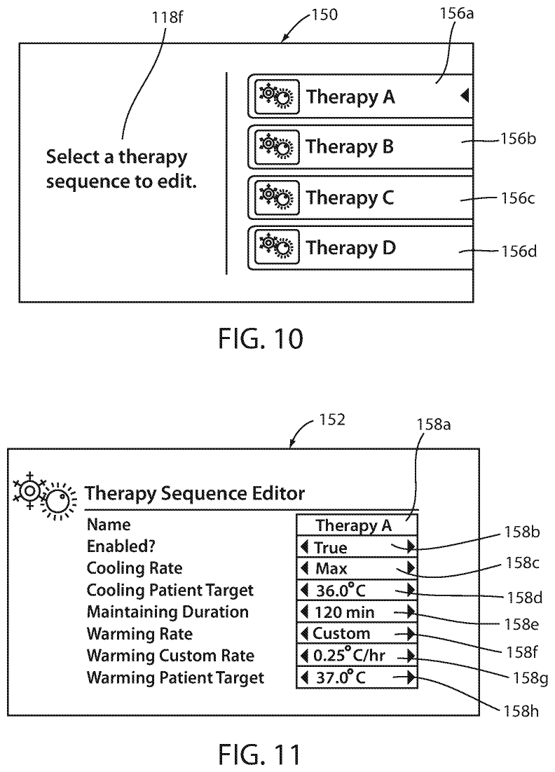

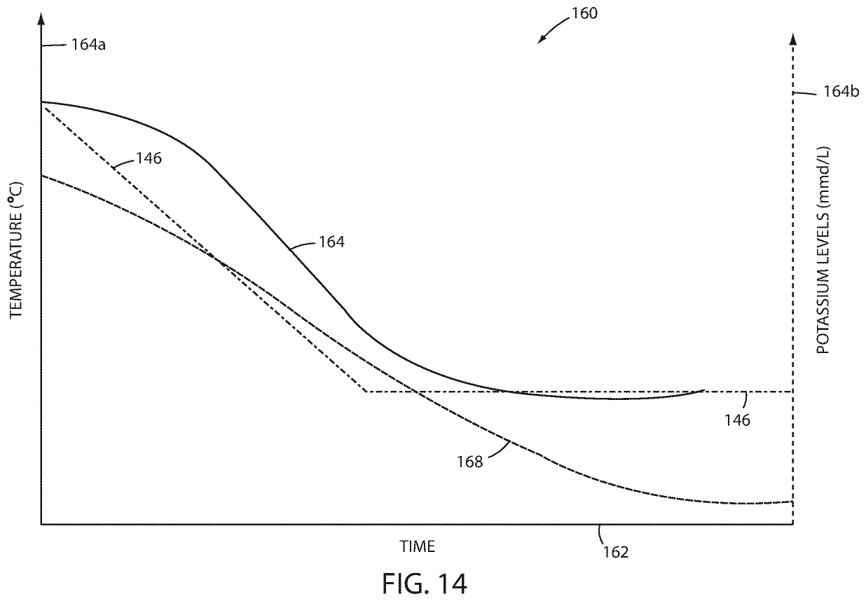

In addition to customizing the alarm conditions 102 and alarm characteristics 104, thermal control unit 22 is adapted, in some embodiments, to enable the user to customize still other aspects. For example, in some embodiments, thermal control unit 22 may be customized such that particular therapies for which it is used are carried out with specific auxiliary sensors. This type of customization is discussed in more detail below with respect to FIG. 9. Additionally, or alternatively, thermal control unit 22 may be configured to allow the users to customize the treatment profile that controller 60 follows during a thermal therapy session, as discussed more below with respect to FIGS. 10 and 11. In still other embodiments, as discussed below with respect to FIGS. 12 and 13, thermal control unit 22 may be customized to include any combination of any of the alarm customizations, auxiliary sensor customizations, therapy profile customizations, and/or other customizations. Still further, thermal control unit 22 may also be customized to display different data on display 88 during a thermal therapy session, as discussed below with respect to FIG. 14.

FIG. 9 illustrates one example of an auxiliary sensor instruction screen 142 that is displayed on display 88 by controller 60 in some embodiments of thermal control unit 22. Auxiliary sensor instruction screen 142 is displayed by controller 60 in order to instruct the user of the type of auxiliary sensor 144 the user should couple to thermal control unit 22 during an upcoming thermal therapy session. As was noted previously, thermal control unit 22 includes one or more auxiliary sensor ports 94 that are adapted to receive outputs from one or more auxiliary sensors 144. The auxiliary sensors 144 provide additional data to controller 60 regarding the patient during a thermal therapy session. In some embodiments, controller 60 is configured to utilize the additional data when deciding how to control heat exchanger 40, while in other embodiments, controller 60 is configured to only record and/or display the data from the auxiliary sensor without using it to control heat exchanger 40.

Thermal control unit 22 is configured to accept a number of different types of auxiliary sensors 144. In one embodiment, thermal control unit 22 is configured to accept one or more of the following types of auxiliary sensors: an end tidal carbon dioxide (ETCO₂) sensor that detects ETCO₂ levels of the patient; a respiration rate sensor that senses the respiration rate of the patient; a blood pressure sensor that detects the blood pressure of the patient; a heart rate sensor that detects the heart rate of the patient; an electrolyte level that detects levels of one or more electrolytes (e.g. potassium) in the patient's blood; a pulse wave velocity sensor that detects the patient's pulse wave velocity; a bioimpedance that detects a bioimpedance of the patient, such as, but not limited to, the bioimpedance at one or more locations on the patient's body in contact with a thermal pad 24; an electrocardiograph sensor that detects an electrocardiogram of the patient; a temperature change sensor that detects a rate of temperature change of the patient; and/or one or more sensors that are integrated into one or more of the thermal pads 24 and that detect characteristics of the thermal pads 24 and/or of the patient.

Auxiliary ports 94 may take on a variety of different forms. In one embodiment, all of the ports 94 (if there are more than one) are of the same type. In another embodiment, thermal control unit 22 includes multiple types of ports. In any of these embodiments, the ports 94 may include, but are not limited to, a Universal Serial Bus (USB) port, an Ethernet port (e.g. an 8P8C modular connector port, or the like), a parallel port, a different (from USB) type of serial port, etc. Ports 94 may also or alternatively be implemented wirelessly, such as via a WiFi transceiver, a Bluetooth transceiver, a ZigBee transceiver, etc.

In some embodiments, any of the auxiliary sensors 144 may be the same as one or more of the auxiliary patient sensors 75 disclosed in commonly assigned PCT patent application number PCT/US2018/066114 filed Dec. 18, 2018, by Applicant Stryker Corporation and entitled THERMAL SYSTEM WITH PATIENT SENSOR(S), the complete disclosure of which is incorporated herein by reference. Alternatively, or additionally, any of the auxiliary sensors 144 may be the same as one or more of the sensors coupled to the control ports 68 disclosed in commonly assigned U.S. patent application Ser. No. 15/820,558 filed Nov. 22, 2017, by inventors Gregory Taylor and entitled THERMAL SYSTEM, the complete disclosure of which is incorporated herein by reference. Still other types of auxiliary sensors may be used with thermal control unit 22.

In at least one embodiment, at least one of the auxiliary ports 94 is adapted to receive sensor readings from an end-tidal carbon dioxide (ETCO₂) sensor coupled to the patient undergoing thermal treatment. In this embodiment, the ETCO₂ sensor is incorporated into a mask, or other apparatus, that captures and/or samples the amount of carbon dioxide in the exhaled breath of the patient. The ETCO₂ sensor may utilize one or more infrared sensors to detect the ETCO₂ levels of the patient, or it may use other technologies for measuring the ETCO₂ levels. The auxiliary port 94 that is dedicated to receiving the ETCO₂ level readings forwards the readings to controller 60. Controller 60, in turn, uses the readings to perform one or more of the following actions, depending upon the particular embodiment: (1) determine an indicator of the patient's metabolic activity, such as by determining the volume carbon dioxide exhaled by the patient over a given time period (e.g. per minute); (2) display the ETCO₂ levels (and/or the indicator) on display 88 of user interface 76; (3) adjust the heating/cooling commands sent to heat exchanger 40; (4) adjust a flow rate of the fluid delivered to thermal pads 24; (5) change one or more of the coefficients discussed above and used in one or more feedback control loops; and/or (6) adjust a reservoir valve that controls the inclusion and exclusion of reservoir 32 from the circulation channel 36 (e.g. controls when fluid circulating in circulation channel 36 is diverted into reservoir 32, rather than bypassing reservoir 32).

In those embodiments where controller 60 is adapted to adjust the heating and/or cooling commands sent to heat exchanger 40 based on the ETCO₂ readings, controller 60 is programmed to increase the cooling (assuming thermal control unit 22 is being used to cool the patient) in response to an increase in ETCO₂ readings, and to do so earlier than it otherwise would in those embodiments where no ETCO₂ readings are utilized. Such increases provide an early indication that the patient is increasing his or her heat output, and by increasing the cooling in response to such increases, thermal control unit 22 is better able to counteract the increased heating, and thereby better maintain the patient at the desired temperature or more quickly bring the patient to the desired temperature. Alternatively, if the ETCO₂ readings decrease, this provides an indication that the patient's heat output is decreasing, and controller 60 is programmed to decrease the cooling (assuming thermal control unit 22 is being used to cool the patient) in response to such decreases in ETCO₂ readings, and to do so earlier than it otherwise would in those embodiments where no ETCO₂ readings are used. This helps avoid overcooling the patient beyond the patient's target temperature. If thermal control unit 22 is being used to warm a patient, rather than cool the patient, controller 60 may be programmed to take the following actions: decrease the heating in response to an increase in ETCO₂ levels, and increase the heating in response to a decrease in ETCO₂ levels.

Controller 60 is configured to display screen 142 after a user selects a particular type of therapy for which thermal control unit 22 is to be used. Once the user selects the particular therapy type, controller 60 consults the auxiliary sensor data 112 stored in memory 80. The auxiliary sensor data 112 indicates the particular type of auxiliary sensor(s) 144 (if any) that are to be used with each type of therapy. As a result, just as the alarms of thermal control unit 22 can be customized according to types of therapy, thermal control unit 22 can be customized so that the users of thermal control unit 22 are instructed to use one or more specific auxiliary sensors 144 during the course of a particular type of thermal therapy session.

For example, if a user is intending to use thermal control unit 22 for treating a patient after a cardiac arrest, thermal control unit 22 can be configured to automatically instruct the user that an auxiliary sensor 144 comprising a peripheral temperature probe be used to measure a peripheral temperature of the patient (in addition to the core patient temperature readings that are provided by patient temperature sensor 86). One example of a peripheral temperature sensor that may be used as an auxiliary sensor 144 with thermal control unit 22 is the peripheral temperature sensor 116 disclosed in commonly assigned PCT patent application number PCT/US2018/064685 filed on Dec. 10, 2018, by applicant Stryker Corporation and entitled THERMAL CONTROL SYSTEM, the complete disclosure of which is incorporated herein by reference. Thermal control unit 22 may also, or alternatively, be modified to include any of the structures and/or functionality of the thermal control unit disclosed in the aforementioned PCT/US2018/064685 application.

The auxiliary sensor data 112 that defines which auxiliary sensor(s) 144 are to be used with which thermal treatment may be entered by authorized users of thermal control unit 22 using user interface 76. As with the alarm customization, the user may need to enter a password or other credentials, in some embodiments, in order to define the auxiliary sensors 144 that are to be used with specific therapies. Alternatively, or additionally, the auxiliary sensor data 112 may be transferred to thermal control unit 22 from another device in any of the manners discussed above, such as but not limited, to the data transfer methods disclosed in the commonly assigned U.S. patent application Ser. No. 15/616,574 filed Jun. 7, 2017, by inventors Gregory Taylor et al. and entitled THERMAL CONTROL SYSTEM, the complete disclosure of which has already been incorporated herein by reference.

It will be understood that, although FIG. 9 illustrates a specific auxiliary sensor 144 (ECG sensor) that is to be used with a particular therapy (therapy A), thermal control unit 22 may be customized such that auxiliary sensor data 112 specifies auxiliary sensor usage according to other parameters besides therapy types. For example, in some embodiments, auxiliary sensor data 112 specifies one or more auxiliary sensors 144 that are to be used for different locations (e.g. different healthcare facility departments) of thermal control unit. Alternatively, or additionally, auxiliary sensor data 112 may specify one or more auxiliary sensors 144 that are to be used when a particular user is using thermal control unit 22. Auxiliary sensor data 112 may therefore correlate auxiliary sensors 144 to any one or more of therapy types, users, and/or locations, depending upon the particular embodiment of thermal control unit 22.

As was noted previously, thermal control unit 22 may also be customized such that controller 60 follows one or more customized thermal therapy profiles. Such thermal therapy profiles specify one or more parameters that controller 60 is to follow during the course of a thermal therapy session and are stored as part of the thermal therapy profile data 106. In those cases in which thermal control unit 22 is used for cooling the patient, the thermal therapy profile often specifies the rate at which the patient is to be cooled, the cooled target temperature for the patient, the length of time the patient is to remain at the cooled target temperature, the rate at which the patient is to be warmed, and the temperature to which the patient is to be warmed back to. In those cases in which thermal control unit 22 is to be used for warming the patient, the thermal therapy profile may specify the rate of warming the patient, the warmed target temperature for the patient, the length of time the patient is to remain warmed, whether the patient is to be cooled, and if so, the rate of the cooling and/or the target temperature of the cooling. Variations and/or additions may be made to the content of both the cooling therapy profiles and the warming therapy profiles.

FIGS. 10 and 11 help illustrate one manner in which a user can customize one or more thermal therapy profiles that are to be followed by controller 60. FIG. 10 illustrates a first therapy editing screen 150 and FIG. 11 illustrates a second therapy editing screen 152. First therapy screen 150 is displayed by controller 60 on display 88 when a user wishes to customize one or more therapy profiles. In some embodiments, the user presses a profile icon, or other control, on touch screen 88 and controller 60 responds by displaying first screen 150. In other embodiments, the user may navigate to first screen 150 in other manners. However arrived at, first therapy editing screen 150 includes a message 118 instructing the user to select a specific therapy profile 156 that he or she wishes to edit.

In the example shown in FIG. 10, first therapy screen 150 lists four therapy profiles 156a-d. It will be appreciated that this number of profiles 156 varied. Further, although FIG. 10 identifies the four different therapy profiles 156 generically (therapy A, therapy B, etc.), in actual use, controller 60 displays a more descriptive term for the various therapies, such as, but not limited to, “cardiac arrest, “neurosurgery,” “fever,” etc. Indeed, in many embodiments, controller 60 is configured to allow the user to assign names of their choosing to the various therapy profiles 156.

Once a user selects one of the therapy profiles 156a-d displayed on first therapy editing screen 150, controller 60 displays a second editing screen 152 that corresponds to the particular therapy profile selected on screen 150. Thus, in the example shown in FIGS. 10 and 11, the user has selected “Therapy A” on first screen 150, and controller 60 is displaying details regarding the profile for “Therapy A” on second screen 152. These details include a plurality of therapy profile settings 158a-h. It will be understood that the particular number of settings 158, as well as their content, may be varied from the example shown in FIG. 11.

Second therapy editing screen 152 (FIG. 11) includes a plurality of arrows positioned adjacent each therapy profile setting 158. The user touches these arrows in order to adjust each of the individual therapy profile settings 158 to a desired state, thereby enabling the user to customize the particular therapy profile that has been selected (e.g. the profile for Therapy A). Therapy profile setting 158a contains the name of the therapy profile and allows the user to assign a name to, and/or edit the name of, the corresponding therapy profile 156. Therapy profile setting 158b allows the user to enable usage of, or disable usage of, the corresponding therapy profile 156. Therapy profile setting 158c allows the user to specify what cooling rate to utilize when cooling the patient, such as, but not limited to, a low cooling rate, a medium cooling rate, and/or a maximum cooling rate.

Therapy profile setting 158d allows the user to specify a target temperature for the patient for the corresponding therapy profile 156. Therapy profile setting 158e allows the user to specify how long the patient is to be maintained at the target temperature specified by setting 158d. Therapy profile setting 158f allows the user to specify whether the warming rate of the patient after the time period specified by setting 158e expires will be one of the standard warming rates of thermal control unit 22, or a customized warming rate. In the example shown in FIG. 11, the user has selected a custom warming rate. Thermal profile setting 158g allows the user to numerically specify the actual warming rate the thermal control unit 22 will attempt to achieve during the warming phase of the thermal therapy session. Finally, thermal profile settings 158h allows the user to specify the temperature that the patient is to be warmed to during the warming phase of the thermal therapy session.

It will be understood that, although FIG. 11 illustrates a therapy profile that involves only a single cooling followed by a single warming, any of the therapy profiles 156 may be customized by the user to include multiple coolings and/or multiple warmings, and that the rates and target temperatures of each of these may be individually specified by the user. Still other modifications can be made to the thermal therapy profiles 156.

Thermal control unit 22 may also be configured to include multiple thermal therapy profiles 156 for the same type of therapy. The multiple therapy profiles 156 may correspond to different users of thermal control unit 22 and/or different locations of thermal control unit 22. Thus, for example, a user may create a first thermal therapy profile 156 that is used for treating a cardiac arrest patient when a first clinician is treating the patient, a second thermal therapy profile 156 that is used for treating a cardiac arrest patient when a second clinician is treating the patient, a third thermal therapy profile 156 that is used for treating a cardiac arrest patient when a third clinician is treating the patient, etc. In addition to, or in lieu of, multiple thermal therapy profiles 156 for the same treatment that differ according to the specific user, thermal control unit 22 may be customized by a user to include multiple thermal therapy profiles 156 for the same treatment that are customized according to the location of the thermal control unit 22, or that are customized according to other parameters.

It will also be understood that thermal control unit 22 is configured to enable the various alarm customizations, user customizations, auxiliary sensor customizations, location customizations, and therapy profile customizations discussed above to be combined in any desired manner. Controller 60 stores such groupings of alarm, user, auxiliary sensor, location, and therapy profile customizations as records within memory 80. Several illustrative examples of such records are shown in FIGS. 12 and 13 and discussed further below.

FIG. 12 illustrates three therapy customization records 170a-c that may be stored in memory 80. Therapy customization records 170a-c are identified according to the therapy they correspond to. That is, therapy customization record 170a corresponds to therapy A; therapy customization records 170b corresponds to therapy B; and therapy customization record 170c corresponds to therapy C. Each customization record includes one or more sets of data. In the example of FIG. 12, each customization record 170a-c includes a set of alarm conditions 172, a set of alarm characteristics 174, a set of auxiliary sensors 176, a set of therapy profiles 178, a set of users 182, and a set of locations 184. Each one of these sets 172, 174, 176, 178, 182, and 184 may contain multiple items, a single item, or it may be an empty set. Further, each customization records 170 contains its own set of these sets 172, 174, 176, 178, 182, and 184, and the set of sets for each customization record 170 may be the completely the same as, completely different from, or partially similar and partially different from the set of sets in the other customization records. For example, customization record 170a contains sets 172a, 174a, 176a, 178a, 182a, and 184a, and customization records 170b contains sets 172b, 174b, 176b, 178b, 182b, and 184b, and set 172a may be the same as, completely different from, or partially the same as and partially different from set 172b. The same is true of sets 174a and 174b, as well as sets 176a and 176b, sets 178a and 178b, and so on.

Controller 60 consults customization record 170a when the user selects therapy type A, such as via screen 136 or another screen. In response to the user's selection of therapy type A, controller 60 implements the specific alarm conditions 102 that are contained within alarm condition set 172a of record 170a and utilizes those alarm conditions during the upcoming therapy session. Controller 60 also uses the alarm characteristics 104 that are contained within the set of alarm characteristics 174a stored in record 170a. Controller 60 further instructs the user to use the one or more auxiliary sensors 144 that are contained within set 176a. Controller 60 also uses the therapy profile 156 that is contained within set 178a. Still further, controller 60 will use any the data contained sets 172a, 174a, 176a, and 178a only if the current user matches a user contained within set 182a and only if the current location matches a location contained within location set 184a.

In those instances where there are multiple customization records 170 for a particular therapy, but different versions of that therapy depending upon the user and/or the location of thermal control unit 22, controller 60 prompts the user for more information in order to identify the current user and/or current location. If any users are excluded from a user set 182, controller 60 prompts the user to identify the current user so that controller 60 can determine which corresponding record 170 should be used for the upcoming therapy session. Similarly, if any locations are excluded from a location set 184, controller 60 prompts the user to identify the current location so that controller 60 can determine which corresponding record should be used for the upcoming therapy session.

Controller 60 therefore allows a user to customize not only the alarms (both conditions and characteristics) for a particular therapy, but also the auxiliary sensors that are to be used for that therapy and the therapy profile that is to be used for that therapy. Still further, if desired, controller 60 can further customize any one or more of these items for that particular therapy based on the particular user who is using thermal control unit 22 and/or the location of thermal control unit 22. In this manner, for example, a particular user can, after inputting location information, user information, and therapy type information, have any or all of the alarms, auxiliary sensors, and/or therapy profiles customized to that particular user for that particular location and that particular therapy. Another user who uses thermal control unit 22 for the same therapy in the same location, however, may utilize a different customization record 170, and therefore may use different alarm conditions, different alarm characteristics, a different therapy profile, and/or a different auxiliary sensor (if any).

FIG. 13 illustrates another example of a set of customization records 180a-c that are arranged according to users, rather than according to therapy types, as with customization records 170a-c of FIG. 12. User customization records 180a-c contain sets of data 172, 174, 176, 178, and 184 that may or may not be the same as the sets 172, 174, 176, 178, and/or 184 of customization records 170. Thus, for example, alarm condition set 172d of user customization record 180a may be the same as, different from, or partially the same as, alarm condition set 172a of customization record 170a. This is likewise true for the other sets of data of record 180a when compared to records 170a.

User customization records 180a-c are used by controller 60 in some embodiments after controller 60 determines the identity of the user (either automatically in any of the manners discussed above, or manually by having the user identify himself or herself). Once controller 60 has determined the user's identity, controller 60 automatically implements the set of alarm conditions 172, alarm characteristics 174, auxiliary sensor types 176, and therapy profile 178 for that particular user. Controller 60 also checks a set of therapies 188 and the location set 184 to determine if any of the possible therapies and/or possible locations have been excluded from these sets. If so, controller 60 prompts the user to input a specific therapy and/or a specific location and utilizes the specific record 180 corresponding to that particular user and that particular location.

It will be understood that there are still other ways of arranging and storing records 170 and 180 besides the therapy-based records 170 of FIG. 12 and the user-based records 180 of FIG. 13. However arranged, the sets of alarm conditions 172 of the various records are stored with the alarm condition data 102 in memory 80; the sets of alarm characteristics 174 are stored with the alarm characteristics data 104; the sets of auxiliary sensor types 176 are stored with the auxiliary sensor data 112; the therapy profiles 178 are stored with the therapy profile data 106; the sets of users 182 are stored with the user data 108; the sets of locations 184 are stored with the location data 110; and the sets of therapy types 188 are stored in memory 80 either as part of the therapy profile data 106 or separately therefrom.

By saving customization records in memory 80, the user does not need to manually re-enter customization data every time he or she uses thermal control unit 22. Instead, the user merely enters whatever criteria are necessary to identify the set of customized parameters he or she desires (or in some cases, the criteria is detected automatically, such as location or user identity). Such criteria may involve any one or more of the user's identity, the location of thermal control unit 22, and/or the therapy to which thermal control unit 22 is going to be used. Once the criteria is entered or detected, controller 60 searches through the customization records and automatically implements the customized parameters that correspond to the entered or detected criteria.

In some embodiments, controller 60 is configured to display the readings from the auxiliary sensor 144 on display 88 along with patient temperature readings and other data gathered during the thermal therapy session. FIG. 14 illustrates one example of a graph 160 that may be displayed on display 88 by controller 60 when at least one of the auxiliary sensor ports 94 is coupled to an auxiliary sensor 144. Graph 160 includes an X-axis 162 that corresponds to time, a first Y-axis 164a that correspond to temperature, and a second Y-axis 164b that corresponds to potassium levels of the patient. Graph 160 further includes a plot of the patient's temperature readings 164, a plot of the target temperature 146 for the patient, and a plot of the patient's potassium levels 168. The patient's temperature readings for plot 164 come from patient temperature sensor 86. The target temperature plot 146 is taken from a corresponding therapy profile 156 or manually entered by a user. The plot of the patient's potassium levels 168 comes from an auxiliary sensor 144 that is adapted to measure the patient's potassium levels.

Controller 60 is configured to display additional readings from additional, or different, auxiliary sensors 144 besides the potassium level sensor shown in FIG. 14. Further, the user can select which auxiliary sensors 144 he or she wishes to have readings of plotted on graph 160, as well as when to add such readings or when to remove such readings from graph 160. Still further, controller 60 is configured to allow a user to add or remove additional data to or from graph 160 besides the readings from the one or more auxiliary sensors 144. Such additional data includes, but is not limited to, any of the following event data: information regarding the delivery of medication (including type of medication, amount, and/or time of delivery); the onset and/or termination of shivering; the adjustment, relocation, cleaning, and/or replacement of one or more thermal pads 24 on the patient; the adjustment, relocation, cleaning, and/or replacement of a temperature sensor 86; the changing of a setting on thermal control unit 22 (e.g. a rate of heating or cooling, a range of acceptable fluid temperature, etc.); the performance of a maintenance task associated with thermal control unit 22; the detection of an error and/or a patient alert event (e.g. a low potassium level, an elevated blood pressure, a low blood pressure, a low oxygen level, etc.); and/or the flushing a patient's body adjacent a temperature sensor.

Some or all of this event data may be manually entered by the user via user interface 76. Controller 60 may also, or alternatively, be configured to automatically detect one or more of these events and add them to graph 160, such as, but not limited to, the automatic detection of patient shivering, the changing of a setting on thermal control unit 22 (e.g. a target temperature, an acceptable range, a warming or cooling rate, etc.), and/or the performance of a maintenance task. Several manners in which controller 60 and thermal control unit 22 can be configured to automatically detect patient shivering are disclosed in commonly assigned U.S. patent application Ser. No. 15/820,558 filed Nov. 22, 2017, by inventors Gregory S. Taylor et al. and entitled THERMAL SYSTEM, the complete disclosure of which is incorporated herein by reference. Another manner of automatically detecting shivering, or the possibility of shivering, includes monitoring the End Tidal Carbon Dioxide (ETCO₂) levels of the patient while undergoing thermal treatment and looking for increases in the patient's metabolism that are indicative of the patient's body expending additional metabolic effort to stay warm. In some embodiments, thermal control unit 22 is configured to process such ETCO₂ readings and issue a notification and/or alert to the caregiver if shivering is detected. Still other manners of detecting shivering can, of course, be used.

Controller 60 is further adapted, in at least some embodiments, to allow the user to customize what data is displayed on graph 160, including the manner in which the data is displayed (e.g. in what units, whether overlaid on top of the patient temperature readings or spaced from these readings, etc.). As with the other customized data discussed here, the data displayed on graph may be customized according to user, location, and/or therapy type, and this customized data may be stored as within one or more customization records 170, 180, etc. so that the display of data is automatically customized to the user's preferences after the relevant data (e.g. location, user, etc.) has been entered or determined.

Other examples of the type of information that may be displayed on graph 160, and/or other examples of the form in which graph 160 may be constructed include the graphs disclosed in commonly assigned U.S. patent application Ser. No. 16/222,004 filed Dec. 17, 2018, by inventors Gregory S. Taylor et al. and entitled THERMAL SYSTEM WITH GRAPHICAL USER INTERFACE, the complete disclosure of which is incorporated herein by reference.

As was noted previously, in some embodiments, thermal control unit 22 is configured to use the readings from one or more auxiliary sensors 144 when controlling heat exchanger 40 and the temperature of the fluid delivered to thermal pads 24. In such embodiments, controller 60 may utilize the readings of the auxiliary sensors 144 for this purpose in any of the manners that the controller disclosed in the commonly assigned PCT patent application number PCT/US2018/066114 uses the auxiliary sensors 75 disclosed therein. Still other manners of using the auxiliary sensors 144 for controlling the temperature of the circulating fluid may also or alternatively be used.

When thermal control unit 22 is configured to receive an auxiliary sensor 144 that is specifically designed to monitor a patient's potassium levels, the auxiliary sensor 144 may be an ECG sensor that outputs its readings to controller 60. In such cases, controller 60 is programmed to utilize these ECG readings to determine the patient's potassium levels. This programming may be accomplished in any of the manners disclosed in the following documents: (1) Cristian Corsi et al., “Innovative Solutions in Health Monitoring at Home: The Real-Time Assessment of Serum Potassium Concentration from ECG,” ICOST 2012, LNCS 7251, pp. 116-123, (2012); (2) Cristiana Corsi et al., “Noninvasive quantification of blood potassium concentration form ECG in hemodialysis patients,” Scientific Reports, 7:42492, DOI: 10.1038 (Feb. 15, 2017); and/or (3) Zachi Attia et al., “Novel Bloodless Potassium Determination Using a Signal-Processed Single-Lead ECG,” Journal of the American Heart Association, 5:e002746, D01:1161 (2016). Still other manners of determining the patient's potassium levels in a non-invasive manner may also or alternatively be used.

Still further, in some embodiments, thermal control unit 22 is configured to receive blood from the patient and heat or cool the blood. In such embodiments, thermal control unit 22 may be configured to automatically test the patient's blood that is passing through thermal control unit 22 for the patient's potassium levels. In such embodiments, thermal control unit does not necessarily need an auxiliary sensor 144 to determine the patient's potassium levels. Several examples of different manners in which thermal control unit 22 can be modified to heat and/or warm a patient's blood directly are disclosed in commonly assigned PCT patent application number PCT/US2018/064685 filed on Dec. 10, 2018, by application Stryker Corporation and entitled THERMAL CONTROL SYSTEM, the complete disclosure of which is incorporated herein by reference.

It will be understood by those skilled in the art that thermal control unit 22 may be modified in a number of ways from the manner in which it has been described above. For example, although thermal control unit 22 has been described above as incorporating the following five general functions of (1) customizing alarm configurations (conditions, characteristics), (2) customizing auxiliary sensor 144 usage, (3) customizing therapy profiles, (4) displaying auxiliary sensor data on graph 160, and (5) customizing what data is displayed on graph 160, it will be understood by those skilled in the art that thermal control unit 22 may be modified to omit one or more of these general functions (and/or any of the other functions described herein). Thus, for example, in some embodiments, thermal control unit 22 is configured to allow a user to customize the alarm settings, but does not include the ability to suggest an auxiliary sensor 144 (or may not even include an auxiliary sensor port 94). As another example, in some embodiments, thermal control unit 22 does not include any customization features, but instead allows a user to graph both the patient temperature readings and the auxiliary sensor on the same screen and/or same graph. Still other combinations of the functions described herein may be implemented in thermal control unit 22.

A number of other modifications to thermal control unit 22 are also possible beyond those disclosed herein. For example, in any of the embodiments disclosed herein, thermal control unit 22 may be modified to include the report-generating features, and/or the user screen customization features, disclosed in commonly assigned U.S. patent application Ser. No. 62/868,098, filed Jun. 28, 2019, by inventors Gregory Taylor et al. and entitled THERMAL SYSTEM WITH IMPROVED USER INTERFACE, the complete disclosure of which is incorporated herein by reference. If the report-generating feature of this application is included within thermal control unit 22, thermal control unit 22 may be further configured to allow the user to customize the therapy reports generated by thermal control unit 22, and the parameters defining the customized report generation may be stored as part of one or more customization records 170, 180, etc. In this manner, the user may customize the contents of the reports based on the particular user, location, and/or therapy type, or in still other manners.

In some embodiments, thermal control unit 22 includes a flow meter at each fluid inlet 62, such as the flow meters 160 disclosed in commonly assigned U.S. patent application Ser. No. 16/222,004 filed Dec. 17, 2018, by inventors Gregory S. Taylor et al. and entitled THERMAL SYSTEM WITH GRAPHICAL USER INTERFACE, the complete disclosure of which is incorporated herein by reference. Regardless of which of these sets of flow meters thermal control unit 22 includes, the outputs of the flow meters are forwarded to controller 60 and controller 60 uses them to determine if an alarm condition is present.

It will also be understood by those skilled in the art that thermal control unit 22 may be additionally and/or alternatively modified to incorporate any of the temperature overshoot reduction methods, structures, and/or algorithms disclosed in commonly assigned U.S. patent application Ser. No. 62/610,319 filed Dec. 26, 2017, by inventors Gregory Taylor et al. and entitled THERMAL SYSTEM WITH OVERSHOOT REDUCTION, the complete disclosure of which is incorporated herein by reference. Additionally or alternatively, thermal control unit 22 may use any of the data and algorithms disclosed in U.S. patent application Ser. No. 62/610,334 filed Dec. 26, 2017, by inventors Christopher Hopper et al. and entitled THERMAL CONTROL SYSTEM when determining when a patient's core temperature will reach its target temperature, and/or when to transition from heating the circulating fluid to cooling the circulating fluid, and vice versa, in order to reduce overshoot. This '334 application is hereby incorporated herein by reference in its entirety.

Additionally, it will be understood that thermal control unit 22 may be implemented to include any of the physical and/or functional aspects of the commercially available Altrix™ Precision Temperature Management System manufactured and sold by Stryker Corporation of Kalamazoo, Mich., many details of which are described in the Operations Manual for the Altrix™ Precision Temperature Management System (doc. 8001-009-001 Rev. G), published in 2016, the complete disclosure of which is also incorporated herein by reference.

Various other alterations and changes beyond those already mentioned herein can be made to the above-described embodiments. This disclosure is presented for illustrative purposes and should not be interpreted as an exhaustive description of all embodiments or to limit the scope of the claims to the specific elements illustrated or described in connection with these embodiments. For example, and without limitation, any individual element(s) of the described embodiments may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Any reference to claim elements in the singular, for example, using the articles “a,” “an,” “the” or “said,” is not to be construed as limiting the element to the singular.

What is claimed is:

1. A thermal control unit for controlling a patient's temperature during a thermal therapy session, the thermal control unit comprising:
a fluid outlet adapted to fluidly couple to a fluid supply line;
a fluid inlet adapted to fluidly couple to a fluid return line;
a circulation channel coupled to the fluid inlet and the fluid outlet;
a pump for circulating fluid through the circulation channel from the fluid inlet to the fluid outlet;
a heat exchanger adapted to add or remove heat from the fluid circulating in the circulation channel;
a fluid temperature sensor adapted to sense a temperature of the fluid;
a patient temperature sensor port adapted to receive patient temperature readings from a patient temperature sensor;
an auxiliary input adapted to receive an output from an auxiliary sensor;
a display; and
a controller adapted to control the heat exchanger in order to control the patient's temperature, the controller further adapted to display an indication on the display identifying a type of auxiliary sensor a user should couple to the auxiliary input in order to carry out the thermal therapy session.

2. The thermal control unit of claim 1 wherein the auxiliary sensor is adapted to detect at least one of the following characteristics of the patient: an end tidal carbon dioxide (ETCO₂) level, an oxygen saturation (SpO₂) level, a respiration rate, a blood pressure, a heart rate, an electrolyte level, a pulse wave velocity, a bioimpedance, an electrocardiogram, a rate of temperature change, or a level of potassium in the patient.

3. The thermal control unit of claim 1 further comprising a therapy-type input adapted to receive an input from the user indicating a therapy type for the thermal therapy session, and wherein the controller is adapted to automatically select the type of auxiliary sensor based on the therapy-type input.

4. The thermal control unit of claim 3 wherein the therapy-type input is a control on a user interface of the thermal control unit, and wherein the therapy type is at least one of a cardiac arrest therapy, a neuro-trauma therapy, a neurosurgery therapy, a fever therapy, or a pediatric therapy.

5. The thermal control unit of claim 1 further comprising a location input adapted to receive an input indicating a location of the thermal control unit within a healthcare facility, the location input including a screen on the display in which the user enters the location of the thermal control unit, and wherein the controller is adapted to automatically select the type of auxiliary sensor based on the location input.

6. The thermal control unit of claim 1 further comprising:
a user input adapted to receive user data identifying a user of the thermal control unit, and
a memory containing a default set of alarm conditions;
wherein the controller is further adapted to perform the following:
(i) automatically select the type of auxiliary sensor based on the user data;
(ii) allow a user to customize the default set of alarm conditions;
(iii) display a graph on the display, the graph including both a plot of patient temperature readings from the patient temperature sensor port plotted with respect to time and a plot of auxiliary readings from the auxiliary input plotted with respect to time; and
(iv) use the output from the auxiliary sensor to control the temperature of the circulating fluid.

7. The thermal control unit of claim 6 wherein the set of default alarm conditions includes at least two of the following alarm conditions: a patient temperature sensor disconnection, a high fluid temperature, a low fluid temperature, a low fluid flow rate, a pause in therapy, a low fluid level, a patient temperature deviation, or a patient temperature sensor malfunction; the memory further includes a plurality of alarm characteristics for each of the at least two alarm conditions, the controller is further adapted to allow the user to customize the plurality of alarm characteristics for each of the at least two alarm conditions; and the alarm characteristics include at least two of the following characteristics: an on/off setting, a tone setting, a priority setting, a repeat/non-repeat setting, a delay between repeats setting, an audio pause setting, or a pause duration setting.

8. The thermal control unit of claim 1 further comprising a memory containing a therapy profile, and wherein the controller is configured to follow the therapy profile during the thermal therapy session, to allow a user to customize the therapy profile, and to store the customized therapy profile in the memory; wherein the therapy profile defines at least two of the following: a target temperature for cooling the patient, a cooling rate for the patient, an amount of time the patient is maintained at a temperature, a warming rate for the patient, or a target temperature for warming the patient.

9. A thermal control unit for controlling a patient's temperature during a thermal therapy session, the thermal control unit comprising:
a fluid outlet adapted to fluidly couple to a fluid supply line;
a fluid inlet adapted to fluidly couple to a fluid return line;
a circulation channel coupled to the fluid inlet and the fluid outlet;
a pump for circulating fluid through the circulation channel from the fluid inlet to the fluid outlet;
a heat exchanger adapted to add or remove heat from the fluid circulating in the circulation channel;
a fluid temperature sensor adapted to sense a temperature of the fluid;
a patient temperature sensor port adapted to receive patient temperature readings from a patient temperature sensor;
a memory containing a set of alarm conditions;
a display; and
a controller adapted to control the heat exchanger in order to control the patient's temperature, the controller further adapted to issue an alarm in response to detecting any one of the alarm conditions in the set of alarm conditions, and the controller still further adapted to allow a user to customize the set of alarm conditions.

10. The thermal control unit of claim 9 wherein the controller is adapted to allow the user to customize the set of alarm conditions by adding an alarm condition to, and subtracting an alarm condition from, the set of alarm conditions; and wherein the set of alarm conditions includes at least two of the following alarms: a patient temperature sensor disconnection, a high fluid temperature, a low fluid temperature, a low fluid flow rate, a pause in therapy, a low fluid level, a patient temperature deviation, or a patient temperature sensor malfunction.

11. The thermal control unit of claim 9 wherein the memory further includes a therapy profile and a plurality of alarm characteristics for each alarm condition in the set of alarm conditions, the therapy profile defining at least two of the following: a target temperature for cooling the patient, a cooling rate for the patient, an amount of time the patient is maintained at a temperature, a warming rate for the patient, or a target temperature for warming the patient; the plurality of alarm characteristics including at least two of the following settings: an on/off setting, a tone setting, a priority setting, a repeat/non-repeat setting, a delay between repeats setting, an audio pause setting, or a pause duration setting; and
wherein the controller is further adapted to perform the following:
(i) allow the user to customize the plurality of alarm characteristics for each of the alarm conditions in the set of alarms conditions,
(ii) follow the therapy profile during the thermal therapy session; and
(iii) allow a user to customize the therapy profile and to store the customized therapy profile in the memory.

12. The thermal control unit of claim 11 further comprising an auxiliary input adapted to receive an output from an auxiliary sensor, the auxiliary sensor being adapted to detect at least one of the following characteristics of the patient: an end tidal carbon dioxide (ETCO₂) level, an oxygen saturation (SpO₂) level, a respiration rate, a blood pressure, a heart rate, an electrolyte level, a pulse wave velocity, a bioimpedance, an electrocardiogram, or a rate of temperature change; and wherein the controller is further configured to display an indication on the display identifying a type of auxiliary sensor the user should couple to the auxiliary input in order to carry out the thermal therapy session.

13. The thermal control unit of claim 12 further comprising a therapy-type input adapted to receive an input from the user indicating a therapy type for the thermal therapy session, wherein the therapy-type input is a control on a user interface of the thermal control unit, the therapy type is at least one of a cardiac arrest therapy, a neuro-trauma therapy, a neurosurgery therapy, a fever therapy, or a pediatric therapy, and the controller is adapted to automatically select the type of auxiliary sensor based on the therapy-type input.

14. The thermal control unit of claim 12 further comprising a user input adapted to receive user data identifying a user of the thermal control unit, wherein the controller is adapted to automatically select the type of auxiliary sensor based on the user data and to use the output from the auxiliary sensor to control the temperature of the circulating fluid, and wherein the auxiliary sensor is a potassium sensor adapted to detect a level of potassium in the patient and the controller is adapted to display the potassium level on the display.

15. A thermal control unit for controlling a patient's temperature during a thermal therapy session, the thermal control unit comprising:
a fluid outlet adapted to fluidly couple to a fluid supply line;
a fluid inlet adapted to fluidly couple to a fluid return line;
a circulation channel coupled to the fluid inlet and the fluid outlet;
a pump for circulating fluid through the circulation channel from the fluid inlet to the fluid outlet;
a heat exchanger adapted to add or remove heat from the fluid circulating in the circulation channel;
a fluid temperature sensor adapted to sense a temperature of the fluid;
a patient temperature sensor port adapted to receive patient temperature readings from a patient temperature sensor;
an auxiliary input adapted to receive an output from an auxiliary sensor;
a display; and
a controller adapted to control the heat exchanger in order to control the patient's temperature, the controller further adapted to display a graph on the display, the graph including both a plot of patient temperature readings from the patient temperature sensor port plotted with respect to time and a plot of auxiliary readings from the auxiliary input plotted with respect to time.

16. The thermal control unit of claim 15 further comprising a second auxiliary input adapted to receive an output from a second auxiliary sensor, and wherein the controller is further adapted to include a plot of readings from the second auxiliary sensor on the graph plotted with respect to time.

17. The thermal control unit of claim 15 wherein the auxiliary sensor is adapted to detect at least one of the following characteristics of the patient: an end tidal carbon dioxide (ETCO₂) level, an oxygen saturation (SpO₂) level, a respiration rate, a blood pressure, a heart rate, an electrolyte level, a pulse wave velocity, a bioimpedance, an electrocardiogram, or a rate of temperature change.

18. The thermal control unit of claim 15 further comprising:
a therapy-type input adapted to receive an input from the a indicating a therapy type for the thermal therapy session, the therapy-type input being a control on a user interface of the thermal control unit, and the therapy type including at least one of a cardiac arrest therapy, a neuro-trauma therapy, a neurosurgery therapy, a fever therapy, or a pediatric therapy; and
a user input adapted to receive user data identifying a user of the thermal control unit;
wherein the controller is further configured to perform the following:
(i) automatically select, based on the therapy type and the user data, a type of auxiliary sensor the user should couple to the auxiliary input in order to carry out the thermal therapy session; and
(ii) display an indication on the display identifying the selected type of auxiliary sensor.

19. The thermal control unit of claim 17 further comprising a memory containing a default set of alarm conditions and a plurality of alarm characteristics for each alarm condition in the default set of alarm conditions, the default set of alarm conditions including at least two of the following alarm conditions: a patient temperature sensor disconnection, a high fluid temperature, a low fluid temperature, a low fluid flow rate, a pause in therapy, a low fluid level, a patient temperature deviation, or a patient temperature sensor malfunction; and the plurality of alarm characteristics including at least two of the following characteristics: an on/off setting, a tone setting, a priority setting, a repeat/non-repeat setting, a delay between repeats setting, an audio pause setting, and a pause duration setting; and
wherein the controller is further configured to allow a user to customize the default set of alarm conditions and the plurality of alarm characteristics.

20. The thermal control unit of claim 17 further comprising a memory containing a therapy profile, the therapy profile defining at least two of the following: a target temperature for cooling the patient, a cooling rate for the patient, an amount of time the patient is maintained at a temperature, a warming rate for the patient, or a target temperature for warming the patient; and
wherein the controller is further configured to follow the therapy profile during the thermal therapy session, to allow a user to customize the therapy profile, and to store the customized therapy profile in the memory.
